(19) United States
(12) Patent Application Publication
(10) Pub. No.: US 2025/0090051 A1
(43) Pub. Date: Mar. 20, 2025

(54) MESH GROUND PLANE FOR AN ANTENNA SYSTEM IN AN ANALYTE MONITORING SYSTEM

(71) Applicant: Dexcom, Inc., San Diego, CA (US)

(72) Inventors: Amit VERMA, Dania Beach, FL (US); Terry T. THOM, Dania Beach, FL (US)

(21) Appl. No.: 18/827,263

(22) Filed: Sep. 6, 2024

Related U.S. Application Data
(60) Provisional application No. 63/582,735, filed on Sep. 14, 2023.

(51) Int. Cl. A61B 5/145; A61B 5/00

(52) U.S. Cl. CPC A61B 5/14532; A61B 5/14503; A61B 5/6833; A61B 5/742; A61B 2560/0209; A61B 2560/045; A61B 2560/0462

(57) ABSTRACT

Aspects of the present disclosure provide techniques for improving a communication range of an analyte sensor system. The analyte sensor system may include an analyte sensor configured to generate analyte data associated with analyte levels of a user of the analyte sensor system, a first conductive portion configured to transmit the analyte data to a communications device, a circuit board configured to operatively connect the analyte sensor with the first conductive portion, and a second conductive portion configured to reflect, away from a body of the user, a portion of power radiated from the first conductive portion associated with transmission of at least the analyte data.

### CROSS-REFERENCE TO RELATED APPLICATIONS

This application claims priority to and benefit of U.S. Provisional Patent Application No. 63/582,735, filed Sep. 14, 2023, which is hereby assigned to the assignee hereof and hereby expressly incorporated by reference herein in its entirety as if fully set forth below and for all applicable purposes.

### TECHNICAL FIELD

The present disclosure relates generally to an electronic device, such as an analyte sensor system for monitoring analyte values of a user.

### BACKGROUND

Diabetes is a metabolic condition relating to the production or use of insulin by the body. Insulin is a hormone that allows the body to use glucose for energy, or store glucose as fat. When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher-than-normal blood sugar level is called “hyperglycemia.” Chronic hyperglycemia can lead to several of health problems, such as cardiovascular disease, cataract and other eye problems, nerve damage (neuropathy), and kidney damage. Hyperglycemia can also lead to acute problems, such as diabetic ketoacidosis—a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce adequate insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

### SUMMARY

Aspects of the present disclosure provide techniques for improving a communication range of an analyte sensor system. The analyte sensor system may include an analyte sensor configured to generate analyte data associated with analyte levels of a user of the analyte sensor system, a first conductive portion configured to transmit the analyte data to a communications device, a circuit board configured to operatively connect the analyte sensor with the first conductive portion, and a second conductive portion configured to reflect, away from a body of the user, a portion of power radiated from the first conductive portion associated with transmission of at least the analyte data.

Additional aspects relate to an antenna system for communicating analyte data. The antenna system may include a first conductive portion operatively coupled to an analyte sensor via a circuit board. The first conductive portion is configured to: receive analyte data associated with analyte levels of a user of an analyte sensor system and transmit the analyte data to a communications device for display to the user. The antenna system may further include a second conductive portion coupled with the circuit board, wherein the second conductive portion is configured to reflect, away from a body of the user, a portion of power radiated from the first conductive portion associated with transmission of at least the analyte data.

Additional aspects relate to an analyte monitoring system. The analyte monitoring system may include a communications device and an analyte sensor system. The analyte sensor system may include an analyte sensor configured to generate analyte data associated with analyte levels of a user of the analyte sensor system and a first antenna system. The first antenna system may include a first conductive portion configured to receive the analyte data from the analyte sensor and to transmit the analyte data to the communications device for display to the user and a second conductive portion configured to reflect, away from a body of the user, a portion of power radiated from the first conductive portion associated with transmission of at least the analyte data. The analyte sensor system may also include a circuit board configured to operatively connect the analyte sensor with the first conductive portion. The communications device may include a second antenna system configured to receive the analyte data from the first antenna system of the analyte sensor system. The communications device may be configured to display the analyte data received from the first antenna system of the analyte sensor system to the user.

Additional aspects relate to a method for wireless communication by an analyte sensor system. The method includes generating analyte data associated with analyte levels of a user of the analyte sensor system, transmitting, using a first conductive portion of an antenna system of the analyte sensor system, the analyte data to a communications device for display to the user, using a second conductive portion of the antenna system to reflect, away from a body of the user, a portion of power radiated from the first conductive portion associated with the transmission of at least the analyte data.

Additional aspects relate to a method for communication between a communications device and an analyte sensor system in an analyte monitoring system. The method includes generating, by the analyte sensor system, analyte data associated with analyte levels of a user of the analyte sensor system, transmitting, by the analyte sensor system using a first conductive portion of a first antenna system of the analyte sensor system, the analyte data to a communications device for display to the user, using, by the analyte sensor system, a second conductive portion of the first antenna system to reflect, away from a body of the user, a portion of power radiated from the first conductive portion associated with the transmission of at least the analyte data, receiving, by the communications device using a second antenna system of the communications device, the analyte data from the first antenna system of the analyte sensor system, and displaying, by the communications device, the analyte data received from the first antenna system of the analyte sensor system to the user.

Other aspects provide: an apparatus operable, configured, or otherwise adapted to perform the aforementioned methods as well as those described elsewhere herein; a non-transitory, computer-readable media comprising instructions that, when executed by one or more processors of an apparatus, cause the apparatus to perform the aforementioned methods as well as those described elsewhere herein; a computer program product embodied on a computer-readable storage medium comprising code for performing the aforementioned methods as well as those described elsewhere herein; and an apparatus comprising means for performing the aforementioned methods as well as those described elsewhere herein. By way of example, an apparatus may comprise a processing system, a device with a processing system, or processing systems cooperating over one or more networks.

The following description and the appended figures set forth certain features for purposes of illustration.

### BRIEF DESCRIPTION OF THE DRAWINGS

Further aspects of the present disclosure will be more readily appreciated upon review of the detailed description of the various disclosed embodiments, described below, when taken in conjunction with the accompanying figures.

FIG. 1 illustrates aspects of an example system that may be used in connection with some embodiments.

FIG. 2 illustrates aspects of an example system that may be used in connection with some embodiments.

FIG. 3A is an example analyte sensor system, in accordance with some embodiments.

FIG. 3B is an example analyte sensor system, in accordance with some embodiments.

FIG. 4 illustrates aspects of an example analyte sensor system, in accordance with some embodiments.

FIG. 5 illustrates aspects of an example analyte sensor system, in accordance with some embodiments.

FIG. 6 illustrates an example radiation pattern of one or more antennas of an analyte sensor system.

FIG. 7 illustrates an example analyte sensor system incorporating a mesh ground plane.

FIG. 8 illustrates an example embodiment in which a mesh ground plane is disposed on an outside of a housing of the analyte sensor system and incorporated into an adhesive patch of the analyte sensor system.

FIG. 9 illustrates an example embodiment in which one or more antennas of the analyte sensor system comprise a J-shaped antenna or a partial spiral antenna.

FIG. 10 illustrates another example embodiment in which the mesh ground plane is arranged inside the housing of the analyte sensor system.

FIG. 11 illustrates another example embodiment in which the mesh ground plane is integrated onto a printed circuit board (PCB) of the analyte sensor system.

FIG. 12 illustrates another example embodiment in which the analyte sensor system includes a plurality of conductive mesh planes.

FIG. 13 depicts a method for wireless communication by an analyte sensor system, according to some embodiments disclosed herein.

FIG. 14 depicts a method for communication between a communications device and an analyte sensor system in an analyte monitoring system, according to some embodiments disclosed herein.

FIG. 15 depicts aspects of an example health monitoring device, according to some embodiments disclosed herein.

FIG. 16 depicts aspects of an example health monitoring device, according to some embodiments disclosed herein.

The figures, described in greater detail in the description and examples below, are provided for purposes of illustration only, and merely depict typical or example embodiments of the disclosure. The figures are not intended to be exhaustive or to limit the disclosure to the precise form disclosed. It should also be understood that the disclosure may be practiced with modification or alteration, and that the disclosure may be limited only by the claims and the equivalents thereof.

### DETAILED DESCRIPTION

Aspects of the present disclosure provide systems, methods, and devices for improving a communication range of an analyte sensor system. For example, analyte sensor systems may be worn by users and are configured to continuously monitor analyte levels of the user. These analyte levels may then be transmitted from the analyte sensor system to a display device (e.g., smart phone) using an antenna system comprising one or more antennas, allowing the user to conveniently track their analyte levels. Certain existing analyte sensor systems are bulky and tend to be uncomfortable to wear. As a result, there is a constant competitive drive to miniaturize analyte sensor systems, for example, to provide better comfort, discreet usage, and/or ease of use to the user.

However, this miniaturization may have negative effects on communication or transmission ranges of the analyte sensor systems, which may result in the display device not receiving the analyte levels of the user. In some cases, these negative effects may be the result of the one or more antennas of the antenna system of the analyte sensor system being positioned closer to the body of the user. For example, when one or more antennas are positioned closer to the body of the user, a significant portion of radio frequency (RF) power radiated by the one or more antennas during analyte data transmission may be absorbed by the body of the user, which significantly reduces the transmission or communication range of the analyte sensor system.

Accordingly, aspects of the present disclosure provide techniques for improving a communication or transmission range of certain analyte sensor systems. In some embodiments, these techniques may involve the use of a conductive mesh ground plane that is arranged between the body of the user and one or more antennas of an analyte sensor system. In some cases, the mesh ground plane may be configured to reflect a portion of RF power that is radiated by the one or more antennas away from the body of the user and towards the display device, thereby increasing the RF power radiated towards the display device and improving the communication range and overall efficiency of the one or more antennas of the antenna system of the analyte sensor system.

The details of some example embodiments of the systems, methods, and devices of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

### System Overview and Example Configurations

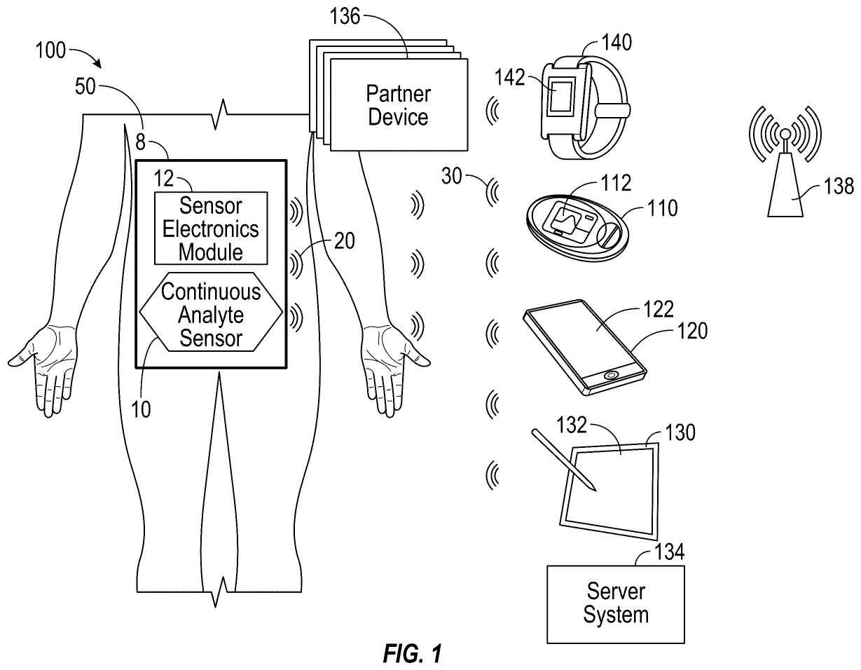

FIG. 1 depicts a system 100 that may be used in connection with embodiments of the present disclosure that involve gathering, monitoring, and/or providing information regarding analyte values present in a user's body, including for example the user's blood glucose values, other analytes, multiple multiplexed or simultaneous measured analytes, or the like. System 100 depicts aspects of analyte sensor system 8 that may be communicatively coupled to display devices 110, 120, 130, and 140, partner devices 136, and/or server system 134.

Analyte sensor system 8 in the illustrated embodiment includes analyte sensor electronics module 12 and analyte sensor 10 associated with analyte sensor electronics module 12. Analyte sensor electronics module 12 may be electrically and mechanically coupled to analyte sensor 10 before analyte sensor 10 is implanted in a user or host. Accordingly, analyte sensor 10 may not require a user to couple sensor electronics module 12 to analyte sensor 10. For example, analyte sensor electronics module 12 may be physically/mechanically and electrically coupled to analyte sensor 10 during manufacturing, and this physical/mechanical and electrical connection may be maintained during shipping, storage, insertion, use, and removal of analyte sensor system 8. As such, the electro-mechanically connected components (e.g., analyte sensor 10 and analyte sensor electronics module 12) of analyte sensor system 8 may be referred to as a “pre-connected” system. Analyte sensor electronics module 12 may be in wireless communication (e.g., directly or indirectly) with one or more of display devices 110, 120, 130, and 140. In addition, or alternatively to display devices 110, 120, 130, and 140, analyte sensor electronics module 12 may be in wireless communication (e.g., directly or indirectly) with partner devices 136 and/or server system 134. Likewise, in some examples, display devices 110-140 may additionally or alternatively be in wireless communication (e.g., directly or indirectly) with partner devices 136 and/or server system 134. Various couplings shown in FIG. 1 can be facilitated with wireless access point (WAP) 138, as also mentioned below.

In certain embodiments, analyte sensor electronics module 12 includes electronic circuitry associated with measuring and processing analyte sensor data or information, including prospective algorithms associated with processing and/or calibration of the analyte sensor data/information. Analyte sensor electronics module 12 can be physically/mechanically connected to analyte sensor 10 and can be integral with (non-releasably attached to) or releasably attachable to analyte sensor 10. Analyte sensor electronics module 12 may also be electrically coupled to analyte sensor 10, such that the components may be electromechanically coupled to one another. Analyte sensor electronics module 12 may include hardware, firmware, and/or software that enables measurement and/or estimation of levels of the analyte in a host/user via analyte sensor 10 (e.g., which may be/include a glucose sensor). For example, analyte sensor electronics module 12 can include one or more of a potentiostat, a power source for providing power to analyte sensor 10, other components useful for signal processing and data storage, and a telemetry module for transmitting data from the sensor electronics module to one or more display devices. Electronics can be affixed to a printed circuit board (PCB) within analyte sensor system 8, or platform or the like, and can take a variety of forms. For example, the electronics can take the form of an integrated circuit (IC), such as an Application-Specific Integrated Circuit (ASIC), a microcontroller, a processor, and/or a state machine.

Analyte sensor electronics module 12 may include sensor electronics that are configured to process sensor information, such as sensor data, and generate transformed sensor data and displayable sensor information. Examples of systems and methods for processing sensor analyte data are described in more detail herein and in U.S. Pat. Nos. 7,310,544 and 6,931,327 and U.S. Patent Publication Nos. 2005/0043598, 2007/0032706, 2007/0016381, 2008/0033254, 2005/0203360, 2005/0154271, 2005/0192557, 2006/0222566, 2007/0203966 and 2007/0208245, all of which are incorporated herein by reference in their entireties.

With further reference to FIG. 1, display devices 110, 120, 130, and/or 140 can be configured for displaying (and/or alarming) displayable sensor information that may be transmitted by analyte sensor electronics module 12 (e.g., in a customized data package that is transmitted to the display devices based on their respective preferences). Each of display devices 110, 120, 130, or 140 can (respectively) include a display such as touchscreen display 112, 122, 132/or 142 for displaying sensor information and/or analyte data to a user and/or receiving inputs from the user. For example, a graphical user interface (GUI) may be presented to the user for such purposes. In embodiments, the display devices may include other types of user interfaces such as voice user interface instead of or in addition to a touchscreen display for communicating sensor information to the user of the display device and/or receiving user inputs. In embodiments, one, some, or all of display devices 110, 120, 130, 140 may be configured to display or otherwise communicate the sensor information as it is communicated from analyte sensor electronics module 12 (e.g., in a data package that is transmitted to respective display devices), without any additional prospective processing required for calibration and/or real-time display of the sensor data.

The plurality of display devices 110, 120, 130, 140 depicted in FIG. 1 may include a custom display device, for example, analyte display device 110, specially designed for displaying certain types of displayable sensor information associated with analyte data received from analyte sensor electronics module 12 (e.g., a numerical value and/or an arrow, in embodiments). In embodiments, one of the plurality of display devices 110, 120, 130, 140 includes a smartphone, such as a mobile phone, based on an Android, IOS, or other operating system, and configured to display a graphical representation of the continuous sensor data (e.g., including current and/or historic data).

As further illustrated in FIG. 1 and mentioned above, system 100 may also include WAP 138 that may be used to couple one or more of analyte sensor system 8, the plurality display devices 110, 120, 130, 140 etc., server system 134, and partner devices 136 to one another. For example, WAP 138 may provide WiFi and/or cellular or other wireless connectivity within system 100. Near Field Communication (NFC) may also be used among devices of system 100 for exchanging data, as well as for performing specialized functions, e.g., waking up or powering a device or causing the device (e.g., analyte sensor electronics module 12 and/or a transmitter) to exit a lower power mode or otherwise change states and/or enter an operational mode. Server system 134 may be used to collect analyte data from analyte sensor system 8 and/or the plurality of display devices, for example, to perform analytics thereon, generate universal or individualized models for glucose levels and profiles, provide services or feedback, including from individuals or systems remotely monitoring the analyte data, and so on.

Partner devices 136, by way of overview and example, can usually communicate (e.g., wirelessly) with analyte sensor system 8, including for authentication of partner devices 136 and/or analyte sensor system 8, as well as for the exchange of analyte data, medicament data, other data, and/or control signaling or the like. Partner devices 136 may include a passive device in example embodiments of the disclosure. One example of partner device 136 may be an insulin pump for administering insulin to a user in response and/or according to an analyte level of the user as measured/approximated using analyte sensor system 8. For a variety of reasons, it may be desirable for such an insulin pump to receive and track glucose values transmitted from analyte sensor system 8 (with reference to FIG. 1 for example). One example reason for this is to provide the insulin pump a capability to suspend/activate/control insulin administration to the user based on the user's glucose value being below/above a threshold value.

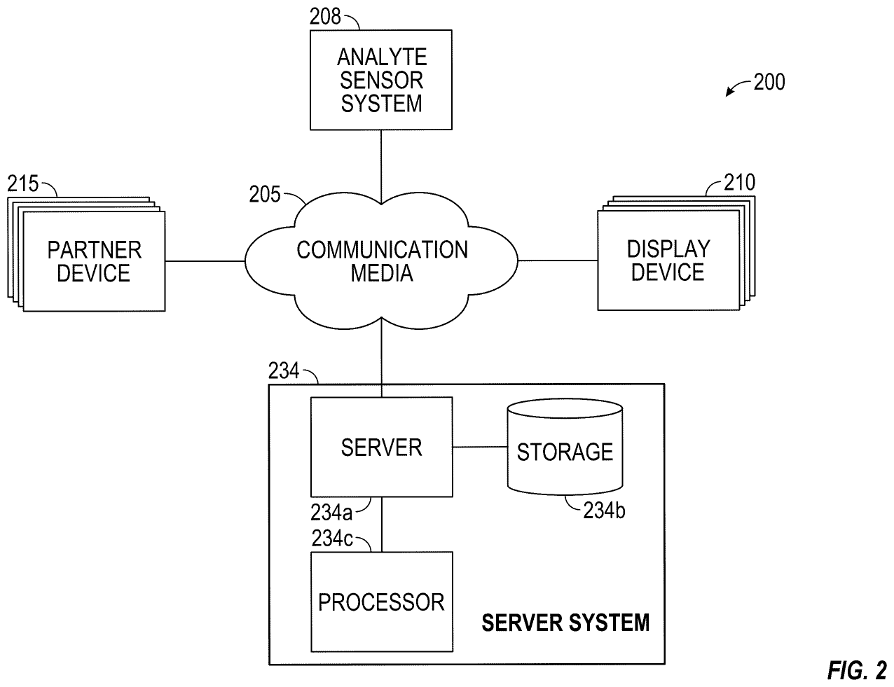

Referring now to FIG. 2, system 200 is depicted. System 200 may be used in connection with implementing embodiments of the disclosed systems, methods, apparatuses, and/or devices, including, for example, aspects described above in connection with FIG. 1. By way of example, various below-described components of FIG. 2 may be used to provide wireless communication of analyte (e.g., glucose) data, for example among/between analyte sensor system 208, display devices 210, partner devices 215, and/or one or more server systems 234, and so on. In some cases, analyte sensor system 208 illustrated in FIG. 2 may be an example of the analyte sensor system 8 illustrated in FIG. 1. Additionally, in some cases, the display devices 210 illustrated in FIG. 2 may be examples of the display devices 110, 120, 130, and 140 illustrated in FIG. 1. Additionally, in some cases, partner devices 215 illustrated in FIG. 2 may be examples of the partner device 136 illustrated in FIG. 1.

As shown in FIG. 2, system 200 may include analyte sensor system 208, one or more display devices 210, and/or one or more partner devices 215. Additionally, in the illustrated embodiment, system 200 includes server system 234, which can in turn include server 234a coupled to processor 234c and storage 234b. Analyte sensor system 208 may be coupled to display devices 210, partner devices 215, and/or server system 234 via communication media 205. Some details of the processing, gathering, and exchanging of data, and/or executing actions (e.g., providing medicaments or related instructions) by analyte sensor system 208, partner devices 215, and/or display device 210, etc., are provided below. Herein, display devices 210, partner devices 215, and server system 234 may be referred to as communications devices and may be configured to communicate with analyte sensor system 208.

Analyte sensor system 208, display devices 210, and/or partner devices 215 may exchange messaging (e.g., control signaling) via communication media 205, and communication media 205 may also be used to deliver analyte data to display devices 210, partner devices 215, and/or server system 234. As alluded to above, display devices 210 may include a variety of electronic computing devices, such as a smartphone, tablet, laptop, wearable device, etc. Display devices 210 may also include analyte display device 110 that may be customized for the display and conveyance of analyte data and related notifications etc. Partner devices 215 may include medical devices, such as an insulin pump or pen, connectable devices, such as a smart fridge or mirror, key fob, and other devices.

In embodiments, communication media 205 may implemented using one or more wireless communication protocols, such as for example Bluetooth, Bluetooth Low Energy (BLE), ZigBee, WiFi, IEEE 802.11 protocols, Infrared (IR), Radio Frequency (RF), 2G, 3G, 4G, 5G, etc., and/or wired protocols and media. It will also be appreciated upon studying the present disclosure that communication media can be implemented as one or more communication links, including in some cases, separate links, between the components of system 200, whether or not such links are explicitly shown in FIG. 2 or referred to in connection therewith. By way of illustration, analyte sensor system 208 may be coupled to display device 210 via a first link of communication media 205 using BLE, while analyte sensor system 208 may be coupled to server system 234 by a second link of communication media 205 using a WiFi communication protocol. In embodiments, a BLE signal may be temporarily attenuated to minimize data interceptions. For example, attenuation of a BLE signal through hardware or firmware design may occur temporarily during moments of data exchange (e.g., pairing).

In embodiments, the elements of system 200 may be used to perform operations of various processes described herein and/or may be used to execute various operations and/or features described herein with regard to one or more disclosed systems and/or methods. Upon studying the present disclosure, one of skill in the art will appreciate that system 200 may include single or multiple analyte sensor systems 208, communication media 205, and/or server systems 234.

As mentioned, communication media 205 may be used to connect or communicatively couple analyte sensor system 208, display devices 210, partner devices 215, and/or server system 234 to one another or to a network. Communication media 205 may be implemented in a variety of forms. For example, communication media 205 may include one or more of an Internet connection, such as a local area network (LAN), a person area network (PAN), a wide area network (WAN), a fiber optic network, internet over power lines, a hard-wired connection (e.g., a bus), DSL, and the like, or any other kind of network connection or communicative coupling. Communication media 205 may be implemented using any combination of routers, cables, modems, switches, fiber optics, wires, radio (e.g., microwave/RF, A M, F M links etc.), and the like. Upon reading the present disclosure, one of skill in the art will recognize other ways to implement communication media 205 for communications purposes and will also recognize that communication media 205 may be used to implement features of the present disclosure using as of yet undeveloped communications protocols that may be deployed in the future.

Further referencing FIG. 2, server 234a may receive, collect, and/or monitor information, including analyte data, medicament data, and related information, from analyte sensor system 208, partner devices 215 and/or display devices 210, such as input responsive to the analyte data or medicament data, or input received in connection with an analyte monitoring application running on analyte sensor system 208 or display device 210, or a medicament delivery application running on display device 210 or partner device 215. As such, server 234a may receive, collect, and/or monitor information from partner devices 215, such as, for example, information related to the provision of medicaments to a user and/or information regarding the operation of one or more partner devices 215. Server 234a may also receive, collect, and/or monitor information regarding a user of analyte sensor system 208, display devices 210, and/or partner devices 215.

In embodiments, server 234a may be adapted to receive such information via communication media 205. This information may be stored in storage 234b and may be processed by processor 234c. For example, processor 234c may include an analytics engine capable of performing analytics on information that server 234a has collected, received, etc. via communication media 205. In embodiments, server 234a, storage 234b, and/or processor 234c may be implemented as a distributed computing network, such as a Hadoop™ network, or as a relational database or the like. The aforementioned information may then be processed at server 234a such that services may be provided to analyte sensor system 208, display devices 210, partner devices 215, and/or a user(s) thereof. For example, such services may include diabetes management feedback for the user.

Server 234a may include, for example, an Internet server, a router, a desktop or laptop computer, a smartphone, a tablet, a processor, a module, or the like, and may be implemented in various forms, including, for example, an integrated circuit or collection thereof, a printed circuit board or collection thereof, or in a discrete housing/package/rack or multiple of the same. In embodiments, server 234a at least partially directs communications made over communication media 205. Such communications may include the delivery of analyte data, medicament data, and/or messaging related thereto (e.g., advertisement, authentication, command, or other messaging). For example, server 234a may process and exchange messages between and/or among analyte sensor system 208, display devices 210, and/or partner devices 215 related to frequency bands, timing of transmissions, security/encryption, alarms, alerts, notifications, and so on. Server 234a may update information stored on analyte sensor system 208, partner devices 215, and/or display devices 210, for example, by delivering applications thereto or updating the same, and/or by reconfiguring system parameters or other settings of analyte sensor system 208, partner devices 215, and/or display devices 210. Server 234a may send/receive information to/from analyte sensor system 208, partner devices 215, and/or display devices 210 in real time, periodically, sporadically, or on an event-drive basis. Further, server 234a may implement cloud computing capabilities for analyte sensor system 208, partner devices 215, and/or display devices 210.

With the above description of aspects of the presently disclosed systems and methods for wireless communication of analyte data, examples of some specific features of the present disclosure will now be provided. It will be appreciated by one of skill in the art upon studying the present disclosure that these features may be implemented using aspects and/or combinations of aspects of the example configurations described above, whether or not explicit reference is made to the same.

### Analyte Data

Referring back to FIG. 1, as mentioned above, in embodiments, analyte sensor system 8 is provided for measurement of an analyte in a host or user. By way of an overview and an example, analyte sensor system 8 may be implemented as an encapsulated microcontroller that makes sensor measurements, generates analyte data (e.g., by calculating values for continuous glucose monitoring data), and engages in wireless communications (e.g., via Bluetooth and/or other wireless protocols) to send such data to remote devices (e.g., display devices 110, 120, 130, 140, partner devices 136, and/or server system 134).

Analyte sensor system 8 may include: analyte sensor 10 configured to measure a concentration or level of the analyte in the host, and analyte sensor electronics module 12 that is typically physically connected to analyte sensor 10 before analyte sensor 10 is implanted in a user. In some cases, the analyte sensor 10 may be a single-analyte sensor or a multi-analyte sensor capable of measuring one or more analytes, such as glucose, lactate, potassium, and the like. In embodiments, analyte sensor electronics module 12 includes electronics configured to process a data stream associated with an analyte concentration measured by analyte sensor 10, in order to generate sensor information that includes raw sensor data, transformed sensor data, and/or any other sensor data, for example. Analyte sensor electronics module 12 may further be configured to generate analyte sensor information that is customized for respective display devices 110, 120, 130, 140, partner devices 136, and/or server system 134. Analyte sensor electronics module 12 may further be configured such that different devices may receive different sensor information and may further be configured to wirelessly transmit sensor information to such display devices 110, 120, 130, 140, partner devices 136, and/or server system 134.

The term “analyte” as used herein is a broad term and is to be given its ordinary and customary meaning to a person of ordinary skill in the art (and is not to be limited to a special or customized meaning), and furthermore refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods is glucose. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1,); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; elements; trace transferring; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

### Analyte Sensor System

As described to above with reference to FIG. 1, in some embodiments, analyte sensor 10 includes a continuous glucose sensor, for example, a subcutaneous, transdermal (e.g., transcutaneous), or intravascular device. In embodiments, such a sensor or device can continuously measure and analyze glucose measurements in the interstitial fluid, blood samples, etc., depending on whether the device is subcutaneous, transdermal, or intravascular. Analyte sensor 10 can use any method of analyte measurement, including for example glucose-measurement, including enzymatic, chemical, physical, electrochemical, spectrophotometric, polarimetric, calorimetric, iontophoretic, radiometric, immunochemical, and the like.

In embodiments where analyte sensor 10 is a glucose sensor, analyte sensor 10 can use any method, including invasive, minimally invasive, and non-invasive sensing techniques (e.g., fluorescence monitoring), or the like, to provide a data stream indicative of the concentration of glucose in a host. The data stream may be a raw data signal, which may be converted into a calibrated and/or filtered data stream that can be used to provide a useful value of glucose to a user, such as a patient or a caretaker (e.g., a parent, a relative, a guardian, a teacher, a doctor, a nurse, or any other individual that has an interest in the wellbeing of the host).

A glucose sensor can be any device capable of measuring the concentration of glucose. According to one example embodiment described below, an implantable glucose sensor may be used. However, it should be understood that the devices and methods described herein can be applied to any device capable of detecting a concentration of an analyte, glucose for example, and providing an output signal that represents the concentration of the analyte, again glucose for example (e.g., as a form of analyte data).

In embodiments, analyte sensor 10 is an implantable glucose sensor, such as described with reference to U.S. Pat. No. 6,001,067 and U.S. Patent Publication No. US-2005-0027463-A1. In embodiments, analyte sensor 10 is a transcutaneous glucose sensor, such as described with reference to U.S. Patent Publication No. US-2006-0020187-A1. In embodiments, analyte sensor 10 is configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007. In embodiments, the continuous glucose sensor includes a transcutaneous sensor such as described in U.S. Pat. No. 6,565,509 to Say et al., for example. In embodiments, analyte sensor 10 is a continuous glucose sensor that includes a subcutaneous sensor such as described with reference to U.S. Pat. No. 6,579,690 to Bonnecaze et al. or U.S. Pat. No. 6,484,046 to Say et al., for example. In embodiments, the continuous glucose sensor includes a refillable subcutaneous sensor such as described with reference to U.S. Pat. No. 6,512,939 to Colvin et al., for example. The continuous glucose sensor may include an intravascular sensor such as described with reference to U.S. Pat. No. 6,477,395 to Schulman et al., for example. The continuous glucose sensor may include an intravascular sensor such as described with reference to U.S. Pat. No. 6,424,847 to Mastrototaro et al., for example.

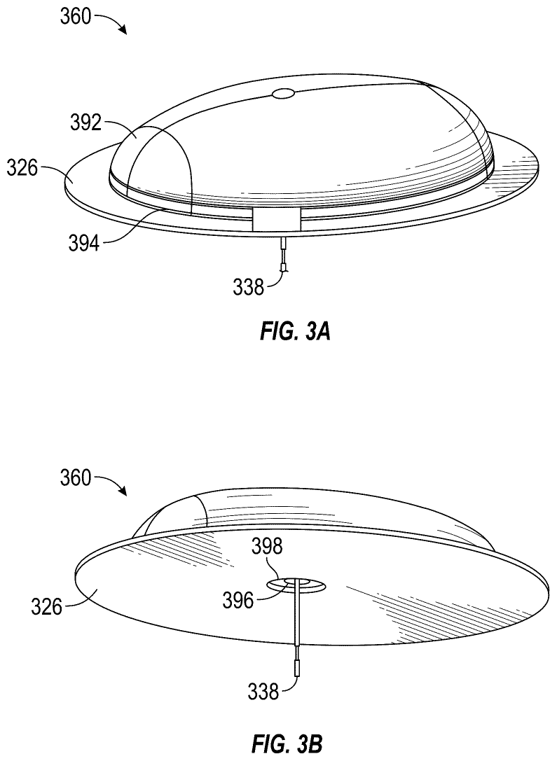

FIG. 3A illustrates a perspective view of an on-skin sensor assembly 360 that may be used in connection with the analyte sensor system 8 of FIG. 1 and/or the analyte sensor system 208 of FIG. 2. For example, on-skin sensor assembly 360 may be or include analyte sensor system 8 and/or analyte sensor system 208. On-skin sensor assembly 360 may include an outer housing with a first, top portion 392 and a second, bottom portion 394. In embodiments, the outer housing may include a clamshell design. On-skin sensor assembly 360 may include, for example, similar components as analyte sensor electronics module 12 described above in connection with FIG. 1, for example, a potentiostat, a power source for providing power to analyte sensor 10, signal processing components, data storage components, and a communication module (e.g., a telemetry module) for one-way or two-way data communication, a printed circuit board (PCB), an integrated circuit (IC), an Application-Specific Integrated Circuit (ASIC), a microcontroller, and/or a processor.

As shown in FIG. 3A, the outer housing may feature a generally oblong shape. The outer housing may further include aperture 396 disposed substantially through a center portion of outer housing and adapted for sensor 338 and needle insertion through a bottom of on-skin sensor assembly 360. In embodiments, aperture 396 may be a channel or elongated slot. On-skin sensor assembly 360 may further include an adhesive patch 326 configured to secure on-skin sensor assembly 360 to skin of the host. In embodiments, adhesive patch 326 may include an adhesive suitable for skin adhesion, for example a pressure sensitive adhesive (e.g., acrylic, rubber-based, or other suitable type) bonded to a carrier substrate (e.g., spun lace polyester, polyurethane film, or other suitable type) for skin attachment, though any suitable type of adhesive is also contemplated. As shown, adhesive patch 326 may feature an aperture 398 aligned with aperture 396 such that sensor 338 may pass through a bottom of on-skin sensor assembly 360 and through adhesive patch 326.

FIG. 3B illustrates a bottom perspective view of on-skin sensor assembly 360 of FIG. 3A. FIG. 3B further illustrates aperture 396 disposed substantially in a center portion of a bottom of on-skin sensor assembly 360, and aperture 398, both adapted for sensor 338 and needle insertion.

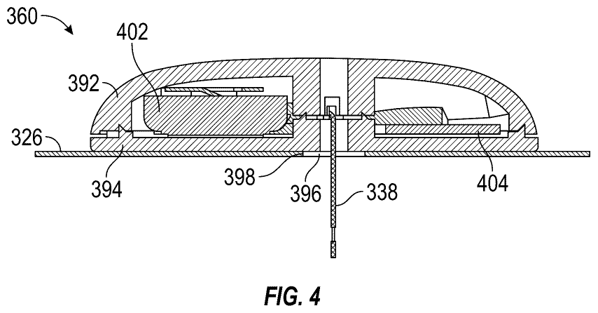

FIG. 4 illustrates a cross-sectional view of on-skin sensor assembly 360 of FIGS. 3A and 3B. FIG. 4 illustrates first, top portion 392 and second, bottom portion 394 of the outer housing, adhesive patch 326, aperture 396 in the center portion of on-skin sensor assembly 360, aperture 398 in the center portion of adhesive patch 326, and sensor 338 passing through aperture 396. The electronics unit, previously described in connection with FIG. 3A, may further include circuit board 404 and battery 402 configured to provide power to at least circuit board 404.

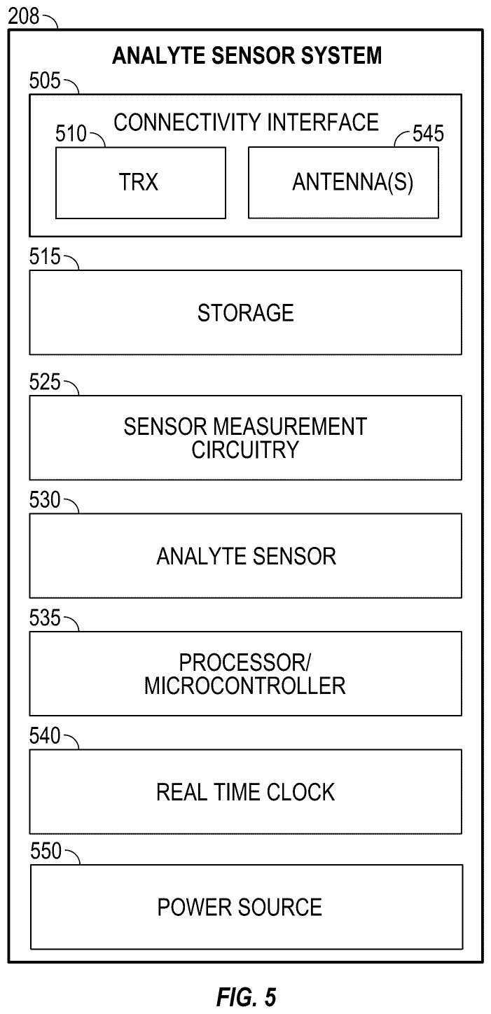

Turning now to FIG. 5, a more detailed functional block diagram of analyte sensor system 208 (discussed above, for example, in connection with FIGS. 1 and 2) is provided. As noted above, the analyte sensor system 208 may be an example of the analyte sensor system 8 illustrated in FIG. 1. As shown in FIG. 5, analyte sensor system 208 may include an analyte sensor 530 (e.g., which may be an example of the analyte sensor 10 illustrated in FIG. 1) coupled to sensor measurement circuitry 525 for receiving, processing, and managing analyte data. Sensor measurement circuitry 525 may be coupled to processor/microcontroller 535. In some embodiments, processor/microcontroller 535 may include one or more processors and may be part of analyte sensor electronics module 12 in FIG. 1. In some embodiments, processor/microcontroller 535 may perform part or all of the functions of sensor measurement circuitry 525 for obtaining and processing analyte data (e.g., sensor measurement values) from the analyte sensor 530. In some embodiments, the processed analyte data may be stored in storage 515, including one or more memories.

Processor/microcontroller 535 may be further coupled to a radio unit or transceiver 510 (e.g., which may be part of analyte sensor electronics module 12 in FIG. 1) for sending sensor and other data and receiving requests and commands and other signaling from an external device, such as display device 310 (referencing FIG. 2 by way of example). In some cases, the transceiver 510 may include logic or circuitry for communicating using different communication protocols, such as Bluetooth, Bluetooth Low Energy (BLE), near-field communication (NFC), and other wireless communication protocols. In some embodiments, the transceiver 510 may be coupled to an antenna system 545 associated with the connectivity interface 505, allowing the analyte sensor system 208 to wirelessly transmit and receive data. For example, the transceiver 510 may be configured to output data for wireless transmission via at least one antenna of the antenna system 545 or may be configured to obtain data that is wirelessly received via at least one of the antennas of the antenna system 545. In some cases, the antenna system 545 may be tuned to a particular frequency depending on a communication protocol used for communicating data. For example, in some embodiments, the antenna system 545 may include one or more antennas tuned for communicating data via a BLE protocol (e.g., tuned to 2.4 gigahertz). In some embodiments, the antenna system 545 may include one or more antennas tuned for communicating data via an NFC protocol (e.g., tuned to 13.56 megahertz).

Analyte sensor system 208, in example implementations, gathers analyte data using the analyte sensor 530 and transmits the same or a derivative thereof to display device 310, partner device 315, and/or server system 334 using the transceiver 510 and antenna system 545. Data points regarding analyte values may be gathered and transmitted over the life of the analyte sensor 530. New measurements and/or related information may be transmitted often enough for a remote device/individual to adequately monitor analyte (e.g., glucose) levels.

It is to be appreciated that some details of the processing, gathering, and exchanging data by analyte sensor system 208, partner devices 315, and/or display device 310 etc. are provided elsewhere herein. It will be appreciated upon studying the present disclosure that analyte sensor system 208 may contain several like components that are described with respect to FIG. 1 or 2, at least for some embodiments herein. The details and uses of such like components may therefore be understood vis-a-vis analyte sensor system 208 even if not expressly described here with reference to FIG. 5.

### Aspects Related to a Mesh Ground Plane for an Analyte Sensor System

Patients with diabetes may benefit from real-time diabetes management guidance that is determined based on a physiological state of the patient. In certain cases, the physiological state of the patient is determined using diagnostics systems, such as an analyte sensor system (e.g., analyte sensor system 8 and/or analyte sensor system 208). In some embodiments, analyte sensor system 208 may be configured to measure analyte levels and inform a patient about the identification and/or prediction of adverse glycemic events, such as hyperglycemia and hypoglycemia. Additionally, the analyte sensor system 208 may be configured to help inform the type of guidance provided to the patient in response to these adverse glycemic events.

For example, the analyte sensor system 208 of FIG. 5 may be worn by a patient and configured to continuously measure the analyte levels of the patient over time using a continuous analyte sensor, such as the analyte sensor 530. The measured analyte levels may then be processed by the analyte sensor system 208 (e.g., by the processor/microcontroller 535) to identify and/or predict adverse events, and/or to provide guidance to the patient for treatment and or actions to abate or prevent the occurrence of such adverse events. Analyte data indicating the patient's analyte levels may then be output to the transceiver 510 of the analyte sensor system 208 for wireless transmission to a communications device via the antenna system 545. In some embodiments, this information may be wirelessly communicated using a Bluetooth low energy (BLE) communication link and antennas included in the analyte sensor system. In some embodiments, the communications device may be at least one of the display devices 210, the partner devices 215, or server system 234 illustrated and described with respect to FIG. 2.

In some embodiments, the antenna system 545 of the analyte sensor system 208 may have a particular radiation pattern. This radiation pattern may represent a relative power radiated by an antenna as a function of a spatial direction away from the antenna. The radiation pattern of the antenna system 545 may be determined based on physical characteristics of the one or more antennas of the antenna system 545, such as size, shape, and orientation, as well as an operating frequency. Power may be radiated by the one or more antennas of the antenna system 545 in a number of lobes or region of the radiation pattern that are bounded by points of equal radiation intensity or power.

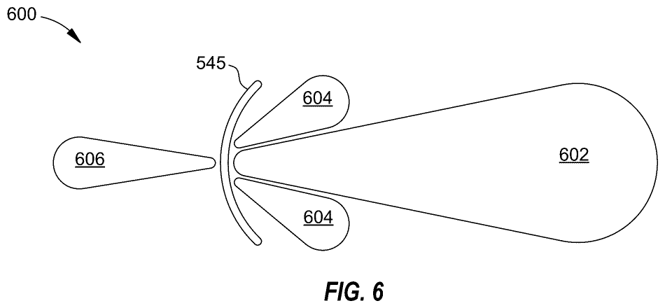

FIG. 6 illustrates an example radiation pattern 600 of the one or more antennas of the antenna system 545. As shown, the radiation pattern 600 includes a main lobe 602, a plurality of side lobes 604, and a back lobe 606. As shown, the main lobe 602 of the radiation pattern 600 represents the spatial direction of maximum radiation intensity or power. In other words, the main lobe 602 is the region of the radiation pattern where most of the radiated energy is concentrated. The main lobe 602 is generally considered to be the most important part of the radiation pattern, as it determines the spatial direction of maximum signal strength and is the spatial direction in which the antenna is pointing. In contrast, the plurality of side lobes 604 are regions of the radiation pattern where radiation intensity is lower than that of the main lobe 602. The plurality of side lobes 604 may occur when the one or more antennas of the antenna system 545 radiates power in spatial directions other than an intended spatial direction. As shown, the plurality of side lobes 604 may occur on either side of the main lobe 602. Similarly, the back lobe 606 is a type of side lobe consisting of regions of the radiation pattern directly opposite the main lobe 602. Both the plurality of side lobes 604 and the back lobe 606 are undesirable as have the potential to cause interference and decrease the overall efficiency of the one or more antennas of the antenna system 545 since power is being radiated in undesirable or un-useful spatial directions.

In some cases, current analyte sensor systems may have a thickness of about 7 millimeters (mm), allowing one or more antennas of these analyte sensor systems to be located at a distance of about 5.5 mm away from a body of a user or patient due to certain design constraints. However, there is a constant competitive drive to miniaturize these analyte sensor systems, for example, to provide better comfort, discreet usage, and/or ease of use to the user. As a result, next-generation analyte sensor systems may be designed to have a thickness of less than half of the current analyte sensor systems. For example, these next-generation analyte sensor systems will have a thickness of about 2.9 mm, reducing the distance between the one or more antennas of these analyte sensor system and the body of the user to approximately 2.2 mm. Moreover, these next-generation analyte sensor systems may be expected to have a longer communication range than current analyte sensor systems. For example, current analyte sensor systems may support a communication range of about 20 feet while next generation analyte sensor systems are expected to support a range of 30 feet or more. In some ideal scenarios, a larger power source (e.g., a larger battery) may provide a higher power communication (with a longer communication range), however, with miniaturization of the next-generation analyte sensor systems such implementations may be prohibitive.

The miniaturization of next generation analyte sensor systems and resulting reduction in distance between the one or more antennas and the body of the user may cause issues in achieving this expected increased communication range. Moreover, the reduction in distance between the one or more antennas and the body of the user may also cause issues related to a bandwidth associated with the sensor antenna. For example, as the one or more antennas are positioned closer to the body of the user, power radiated from a back lobe of the one or more antennas may be absorbed by the body of the user, causing a variation of input impedance, frequency shift and reduced efficiency and gain of the one or more antennas. As a result, due to the miniaturization of next-generation analyte sensor systems, the body of the user may negatively affect a radiation pattern of the one or more antennas of these analyte sensor systems, causing a reduction in communication range between these analyte sensor systems and associated communications devices and leading to poor user experience.

In some cases, one manner of reducing the negative effects associated with miniaturization and antennas located closer to a body of the user may be to use a larger antenna. For example, for some portable computational devices (e.g., smartphones, tablets, and laptop computers), the size of the device may allow for half-wave antennas to be used for wireless communications, which may improve antenna gain, antenna efficiency, frequency response, transmission range, etc. However, the size of next-generation analyte sensor systems may not allow these analyte sensor systems to be equipped with a large antenna (e.g., a half-wave antenna or larger). Instead, the size of these analyte sensor systems may only allow the use of quarter-wave antennas or smaller, which may have lower antenna gain and lower antenna performance and, thus, may not be able to transmit as far as larger antennas. In some cases, rather than using a larger antenna, a radiation pattern of the smaller, quarter-wave antenna may be designed to be directive, allowing radiated power to be concentrated in a particular spatial direction (e.g., a radiation pattern having a main lobe concentrated in a single direction) and improving antenna gain and/or performance of the quarter-wave antenna.

However, while directionality may improve antenna gain and/or performance, directionality may give rise to additional issues. For example, in some cases, the user of an analyte sensor system may continuously move locations throughout the day. These changes in location of the user (as well as the analyte sensor system) may change the direction of the radiation pattern (e.g., in particular the main lobe) of the antenna of the analyte sensor system relative to the communications device, leading to misalignment between the analyte sensor system and communications device. In some cases, these changes in position and misalignment between the analyte sensor system and the communications device may, in turn, lead to obstructions being positioned between the antenna of the sensor system and the communications device, such as the body of the user, another person, a wall, etc. Further, the misalignment and obstructions between the analyte sensor system and communications device may result in degraded communication performance (e.g., reduced throughput, increased latency, reduced transmission range, and/or a lost communication link). In addition to changes in the location of the user, the analyte sensor system may be placed on various different positions on the body of the user (e.g., arm, torso, leg), each of which may be associated with a different antenna efficiency and/or frequency response, causing similar issues with degraded communication performance over time.

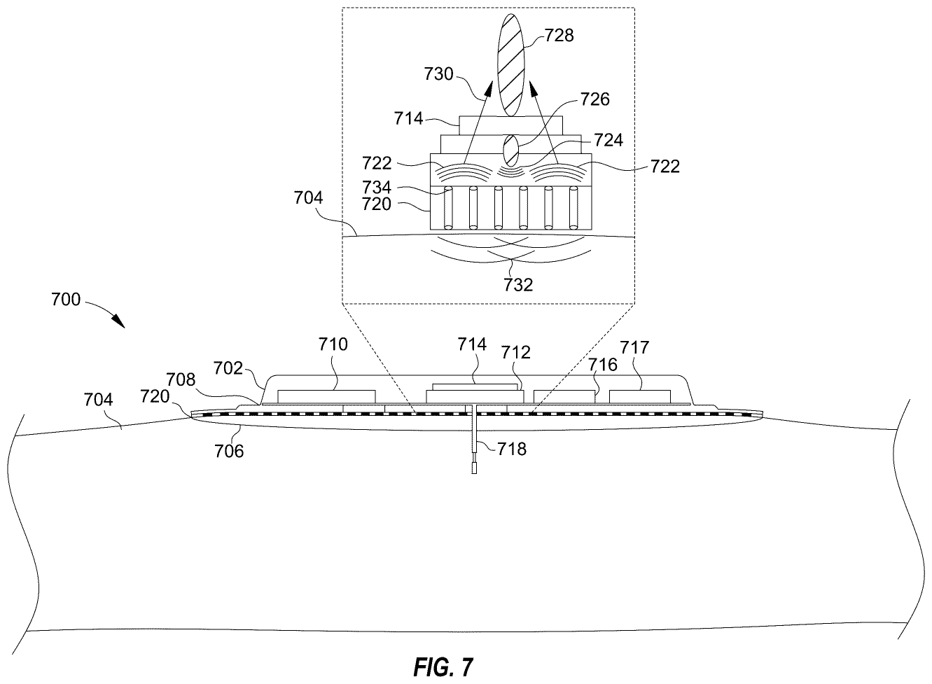

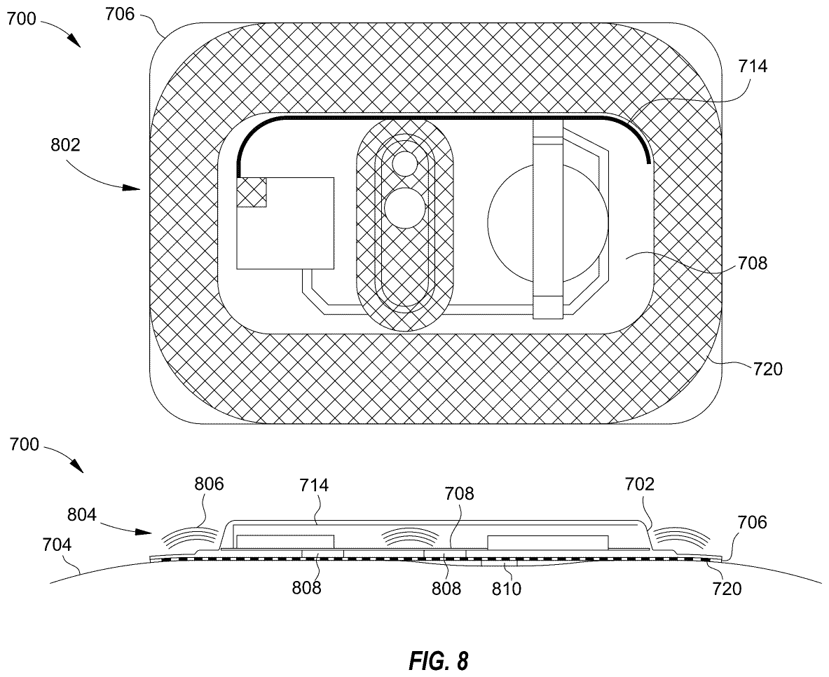

Accordingly, aspects of the present disclosure provide techniques for improving antenna performance of a next-generation analyte sensory system, such as an analyte sensor system that is designed to have a small distance (e.g., below a threshold, such as approximately 5 mm) between one or more antennas included therein and a body of the user. In some embodiments, these techniques may involve the use of a conductive mesh ground plane that is arranged between the body of the user and one or more antennas of an analyte sensor system. One example of this mesh ground plane is illustrated in and described with respect to FIG. 7. Additional embodiments are shown and described with respect to FIGS. 8-12.

### Example Analyte Sensor System and Mesh Ground Plane

FIG. 7 illustrates a side view of an analyte sensor system 700 that includes a mesh ground plane 720. The analyte sensor system 700 may be an example of the analyte sensor system of FIG. 1 and/or the analyte sensor system 208 of FIGS. 2 and 5. As shown, the analyte sensor system 700 includes a waterproof housing 702 that may be adhered to a body of a user 704 using an adhesive patch 706. The waterproof housing 702 may house one or more components of the analyte sensor system 700, including a printed circuit board (PCB) 708, a processor/microcontroller 710 (e.g., including one or more processors), a transceiver 712 coupled with one or more antennas 714, a storage 716 (e.g., including one or more memories), a battery 717, and an analyte sensor 718. In some embodiments, the battery 717 may be configured to power the one or more components of the analyte sensor system 700. In some embodiments, the one or more antennas 714 may comprise a first conductive portion of an antenna system of the analyte sensor system 700. The first conductive portion may comprise a radiative element configured for wirelessly transmitting analyte data of the user 704.

In some embodiments, the PCB 708 may include circuitry for operatively connecting the processor/microcontroller 710, the transceiver 712, the one or more antennas 714, the storage 716, and the analyte sensor 718. In some cases, the processor/microcontroller 710 may be an example of the processor/microcontroller 535 described with respect to FIG. 5, the transceiver 712 may be an example of the transceiver 510 described with respect to FIG. 5, the one or more antennas 714 may be an example of the one or more antennas in the antenna system 545 described with respect to FIG. 5, the storage 716 may be an example of the storage 515 described with respect to FIG. 5, and the analyte sensor 718 may be an example of the analyte sensor 530 described with respect to FIG. 5.

As noted above, the analyte sensor system 700 includes the analyte sensor 718 coupled with sensor measurement circuitry (e.g., sensor measurement circuitry 525 illustrated in FIG. 5), which are configured to generate analyte data associated with analyte levels of the user of the analyte sensor system 700 and provide the analyte data to the processor/microcontroller 710. In some embodiments, the processor/microcontroller 710 may be configured to process the analyte data and provide the processed analyte data to the transceiver 712 for transmission. For example, in some embodiments, after receiving the analyte data from the processor/microcontroller 710, the transceiver 712 may output the analyte data to an antenna feed of the transceiver 712 for wireless transmission to a communications device via the one or more antennas 714 using Bluetooth low energy (BLE) or another wireless communication technology.

In some embodiments, the communications device may be a display device for displaying the analyte data to the user 704. In some cases, the communications device may be an example of one or more of the display devices 210, the partner devices 215, and/or the server system 234 illustrated and described with respect to FIG. 2. In some embodiments, the communications device may include a second antenna system, comprising one or more antennas, configured to receive the analyte data from the first antenna system of the analyte sensor system 700. As noted above, the communications device may be configured to display the analyte data received from the first antenna system of the analyte sensor system 700 to the user 704. In some embodiments, the one or more antennas 714 may be configured to transmit raw sensor data from the analyte sensor 718 to the communications device. In such cases, the communications device may be configured to process the raw sensor data to obtain the analyte levels of the user 704 and to display the analyte levels to the user 704.

Additionally, as shown, the analyte sensor system 700 shown in FIG. 7 includes a mesh ground plane 720. The mesh ground plane 720 may comprise a second conductive portion of the antenna system of the analyte sensor system 700 and may be configured to improve a communication range and overall efficiency of the one or more antennas 714 when transmitting analyte data or other transmissions to the communications device. For example, to improve the communication range and overall efficiency of the one or more antennas 714, the second conductive portion of the mesh ground plane 720 may include a reflective element configured to reflect a first portion 722 (e.g., 80%) of radio frequency (RF) power 724 that is radiated in a back lobe 726 or side lobes of a radiation pattern of the one or more antennas 714 to a main lobe 728 of the one or more antennas 714, as shown at 730, while still allowing a second portion 732 of the radiated RF power 724 radiated in the back lobe 726 or side lobes of the one or more antennas 714 to be absorbed by the body of the user 704.

For example, the mesh ground plane 720 may include a plurality of apertures 734 (or openings) that permit the second portion 732 of the radiated RF power 724 to pass through the mesh ground plane 720 and to be absorbed by the body of the user 704 while also reflecting the first portion 722 of radiated RF power 724 to the main lobe 728 of the one or more antennas 714. In some embodiments, the plurality of apertures 734 may not include cut outs or holes within the mesh ground plane 720 that are needed to fit or secure the mesh ground plane 720 within the housing 702 of the analyte sensor system 700 or to accommodate fitting other components of the analyte sensor system 700 within the housing 702. Rather, in some embodiments, the plurality of apertures 734 may be uniformly or periodically distributed across the mesh ground plane 720, forming a mesh or web-like structure.

In some embodiments, the mesh ground plane 720 helps to resolve an issue of on-body degradation of radiated RF power of the one or more antennas 714 when the one or more antennas 714 are arranged close to the body of the user 704 (e.g., a displacement of less than or equal to 2.9 mm). For example, by reflecting the first portion 722 of radiated RF power 724 of the back lobe 726 of the one or more antennas 714 to the main lobe 728 of the one or more antennas 714, radiated RF power of the main lobe 728 may be increased, thereby increasing a gain of the one or more antennas 714 and a communication range of the one or more antennas 714. In another example, reflecting the first portion 722 of radiated RF power 724 to the main lobe 728 of the one or more antennas 714 may allow the one or more antennas 714 to have a communication range of 30 feet or more while still allowing for the miniaturization of the analyte sensor system 700 discussed above. Additionally, by allowing the second portion 732 of the radiated RF power 724 to be absorbed by the body of the user 704, a bandwidth of the one or more antennas 714 may be improved and/or maintained within a certain communication range due to interfering side lobe(s) being permitted to be absorbed. In some embodiments, the certain communication range may comprise a Bluetooth communication range (e.g., 2.4 GHz), a WiFi communication range, cellular communication range (e.g., for 2G, 3G, 4G, 5G, and/or later generations communications), and/or other communication ranges associated with other wireless standards. It will be appreciated that the plurality of apertures 734 may be tuned or adjusted to provide a maximum bandwidth, efficiency and gain for the one or more antennas 714.

For example, without the mesh ground plane 720, the analyte sensor system 700 may expect an output power of about −18 dBm to −16 dBm due to efficiency degradation of the one or more antennas 714 resulting from antenna radiation absorption by the body of the user. This output power is very poor and results in a communication range of only about 18 to 20 feet. Conversely, when the mesh ground plane 720 is included in the analyte sensor system 700, a power gain of about 6 dBm may be achieved, resulting in an output power of approximately −12 dBm to −10 dBm. This increased output power may allow the communication range of the analyte sensor system 700 to be increased to 30 to 45 feet or more.

In some embodiments, the mesh ground plane 720 may be composed of conductive material that includes a plurality of openings or apertures (e.g., the plurality of apertures 734) arranged across the conductive material. In some embodiments, the conductive material may include at least one of steel, stainless steel, galvanized steel, aluminum, copper, titanium, silver, gold, or any other conductive material. In some cases, the mesh ground plane may be coated with a protective covering to prevent corrosion or degradation of the conductive material. In some embodiments, the mesh ground plane 720 may include an arrangement of interlocking electrically conductive links or segments with various apertures or openings arranged throughout. In some embodiments, the mesh ground plane 720 may be composed of a plurality of conductive filaments woven together into a web-like pattern including multiple apertures or openings arranged throughout. In some cases, the mesh ground plane 720 may be composed of a flexible conductive material, such as a flexible graphite film, that can be implemented on various surfaces (e.g., flat, curved, round, etc.) of the analyte sensor system 700. The density of the graphite film may be 5 times less than the copper film and may be used for antenna design or grounding purposes. Additionally, flexible graphite film may have excellent structure stability and mechanical flexibility.

In some embodiments, an arrangement, size, and/or shape of the plurality of apertures 734 in the mesh ground plane 720 may be configured to tune a bandwidth and reflection response achieved by the mesh ground plane 720 to an operating frequency defined in a wireless communication standard. For example, as a size of the plurality of apertures increases, the bandwidth of the one or more antennas 714 may increase. In contrast, as the size of the plurality of apertures decreases, the bandwidth of the one or more antennas 714 may decrease. For example, a solid ground plane (e.g., a ground plane without apertures) may result in the one or more antennas 714 having a very narrow bandwidth.

In some examples, the mesh ground plane 720 may be designed to achieve a particular and/or preferred antenna bandwidth (>80 MHz) depending on the arrangement, size, and/or shape of the plurality of apertures 734. In some examples, this particular antenna bandwidth may comprise a bandwidth that is compatible with a particular wireless communications standard, such as Bluetooth, 3GPP, IEEE 802.11, etc. For example, the plurality of apertures 734 may each, individually, have a width or diameter of about 0.5 mm to 1 mm and/or have a collective density that ensures a bandwidth of the one or more antennas 714 of the analyte sensor system 700 is greater than or equal to 80 MHz consistent with Bluetooth wireless standards or an operating frequency of the one or more antennas 714. In certain examples, the mesh ground plane 720 may be designed to resonate and/or reflect radiation at a particular operating frequency (or range of frequencies). In some cases, the operating frequency may comprise an industrial, scientific, and medical (ISM) frequency band, such as 2.4 GHz for Bluetooth communications, a frequency or range of frequencies used for WiFi communications, a frequency or range of frequencies used for cellular communications (e.g., 2G, 3G, 4G, 5G, and/or later generations), and/or other frequency ranges for communications based on other wireless standards.

In certain aspects, the mesh ground plane 720 may include one or more mesh portions and/or one or more sheet or plate portions. In other words, the mesh ground plane 720 may be partially formed of conductive mesh and partially formed of a solid sheet or plate. The conductive mesh of the mesh ground plane 720 may cover a portion of a particular side of the analyte sensor system 700 (e.g., a portion of a bottom surface of the analyte sensor system 700) or an entire surface (e.g., the entire bottom surface of the analyte sensor system 700). In some cases, the conductive mesh may cover an area of at least 50% of the bottom surface of the analyte sensor system 700, including a portion of the bottom surface below the one or more antennas 714.

In some aspects, the one or more antennas 714 of the analyte sensor system 700 may be embedded in a sensor socket. In such aspects, a sensor wire of the analyte sensor 718 (e.g., responsible for performing analyte measurements of a user) may be tuned with the one or more antennas 714. Having the sensor wire of the analyte sensor 718 tuned with the one or more antennas 714 may allow for the analyte sensor system 700 to detect a certain state of the sensor wire. For example, if the sensor wire is not properly placed in the device (e.g., due to a missing sensor wire) or is damaged, an antenna response of the one or more antennas 714 may be skewed, allowing the analyte sensor system 700 to detect the damaged or unplaced sensor wire. Additionally, the antenna response of the one or more antennas 714 may be indicative of a particular state associated with the sensor wire, such as the sensor wire operating as expected, the sensor wire is broken, damaged, or missing. Such states associated with the sensor wire may be detected based on a signal strength, signal quality, etc. For example, a reduction in a received signal strength indicator (RSSI) associated with the one or more antennas 714 may indicate a broken or damaged sensor wire.

The mesh ground plane 720 may be implemented in the analyte sensor system 700 in a variety of manners, which are described in greater detail below with respect to FIGS. 8-12. For example, FIG. 8 illustrates an example embodiment in which the mesh ground plane 720 is disposed on the outside of the housing 702 and incorporated into the adhesive patch 706 of the analyte sensor system 700. In some aspects, the adhesive patch 706 may be attached to the outside of the housing 702 of the analyte sensor system 700.

More specifically, for example, FIG. 8 illustrates a bottom view 802 of the analyte sensor system 700 and a cross-sectional view 804 of the analyte sensor system 700. As shown, the mesh ground plane 720 may be incorporated into the adhesive patch 706 that is situated between the body of the user 704 and the one or more antennas 714 of the analyte sensor system 700. As noted above, the mesh ground plane 720 may allow RF radiation, which is emitted from the one or more antennas 714, to be reflected away from the body of the user 704 as shown at 806. As discussed above, this reflection of RF radiation by the mesh ground plane 720 may enhance power of a main lobe (e.g., main lobe 728 illustrated in FIG. 7) of the one or more antennas 714, resulting in an improved transmission/communication range of the one or more antennas 714 of the analyte sensor system 700 and allowing the analyte sensor system 700 to be reduced in size without the negative effects discussed above. As discussed above, in the example of FIG. 8, the mesh ground plane 720 is illustrated as being incorporated into the adhesive patch 706 of the analyte sensor system 700. In some embodiments, for example, the mesh ground plane 720 may be woven into a fabric of the adhesive patch 706 or may be laminated between different layers of the adhesive patch 706. In some embodiments, the mesh ground plane 720 may be arranged between the adhesive patch 706 and the housing 702 of the analyte sensor system 700.

In some embodiments, the mesh ground plane 720 may be electrically coupled to the PCB 708 of the analyte sensor system by a conductive contact 808. For example, by electrically coupling the mesh ground plane 720 with the PCB 708, the mesh ground plane 720 may serve as a ground, allowing energy to travel through ground and to radiate away from the body of the user 704. In some cases, if the mesh ground plane 720 were not to be electrically coupled with the PCB 708, this may lead to a scenario involving a “floating” ground, which may reduce an efficiency associated with the one or more antennas 714. In some cases, the conductive contact 808 may be a thin trace, a wire, a conductive pad, etc.

Additionally, the mesh ground plane 720 may also be electrically coupled to the body of the user 704. In some embodiments, the mesh ground plane 720 may be electrically coupled to the body of the user 704 through direct contact (e.g., the mesh ground plane is situated directly on the body of the user 704). For example, the mesh ground plane 720 may be disposed on a surface of the adhesive patch 706 configured to be attached to the body of the user 704. In other embodiments, the mesh ground plane 720 may not be in direct contact with the body of the user 704, instead being covered in an adhesive (e.g., glue) of the adhesive patch 706 and coupled to the body of the user 704 by a (relatively small) conductive contact 810 included in the adhesive patch 706. In some embodiments, the conductive contacts 808 and 810 may be or may include a conductive glue, a conductive sponge, and/or a conductive material (e.g., gold or copper), for example.

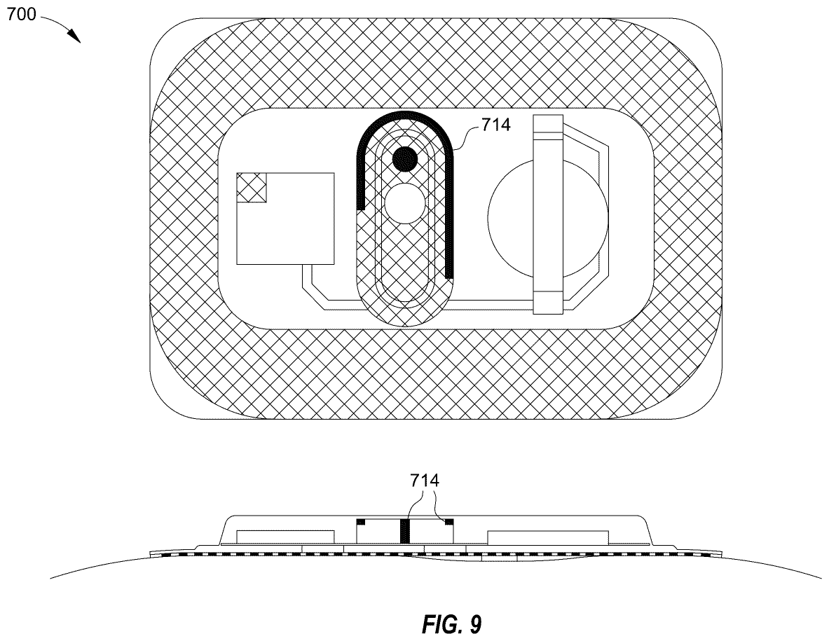

The analyte sensor system 700 may include any of a variety of antenna architectures. For example, as shown in FIG. 8, the one or more antennas 714 of the analyte sensor system 700 may include an L-shaped antenna, which may be an inverted-L. In other embodiments, as shown in FIG. 9, the one or more antennas 714 of the analyte sensor system 700 may include a J-shaped antenna or a partial spiral antenna. In other embodiments, the one or more antennas 714 of the analyte sensor system 700 may include a patch antenna, a slot antenna, a spiral antenna, an inverted-F antenna (e.g., including a planar inverted-F antenna (PIFA) and/or a meandered inverted-F antenna (MIFA)), an inverted-L antenna, a quarter-wave monopole, etc.

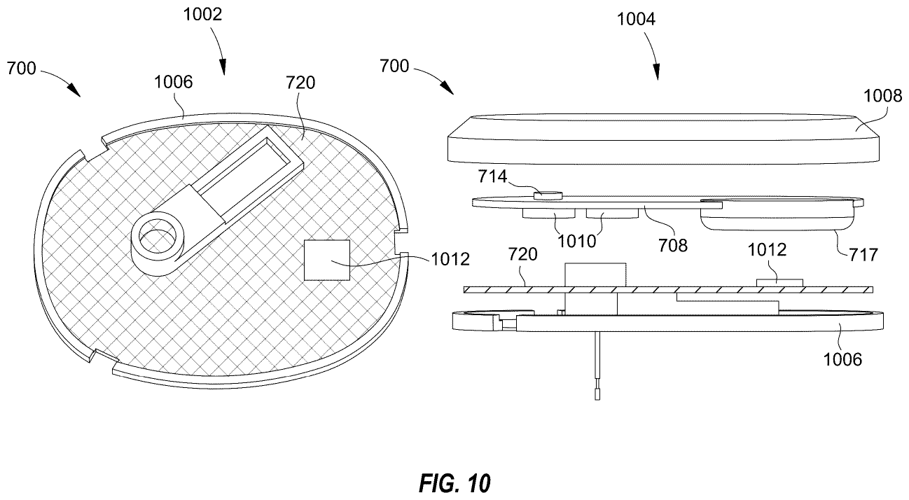

FIG. 10 illustrates another embodiment in which the mesh ground plane 720 is arranged inside a housing of the analyte sensor system 700. For example, FIG. 10 illustrates an isometric view 1002 of a bottom portion 1006 of the housing (e.g., waterproof housing 702 illustrated in FIG. 7) of the analyte sensor system 700 and a cross-sectional exploded view 1004 of the analyte sensor system 700. As shown in FIG. 10, the mesh ground plane 720 is arranged between the bottom portion 1006 of the housing of the analyte sensor system 700 and the PCB 708 of the analyte sensor system 700. Further, as shown, the one or more antennas 714 of the analyte sensor system 700 may be arranged on a top side of the PCB 708. In some embodiments, the one or more antennas 714 may be arranged on a bottom side of the PCB 708 between the PCB 708 and the mesh ground plane 720. As shown, a conductive contact 1010 may electrically couple the mesh ground plane 720 to the PCB 708. Additionally, as shown, a conductive contact 1012 may be coupled between the mesh ground plane 720 and a ground contact of the battery 717.

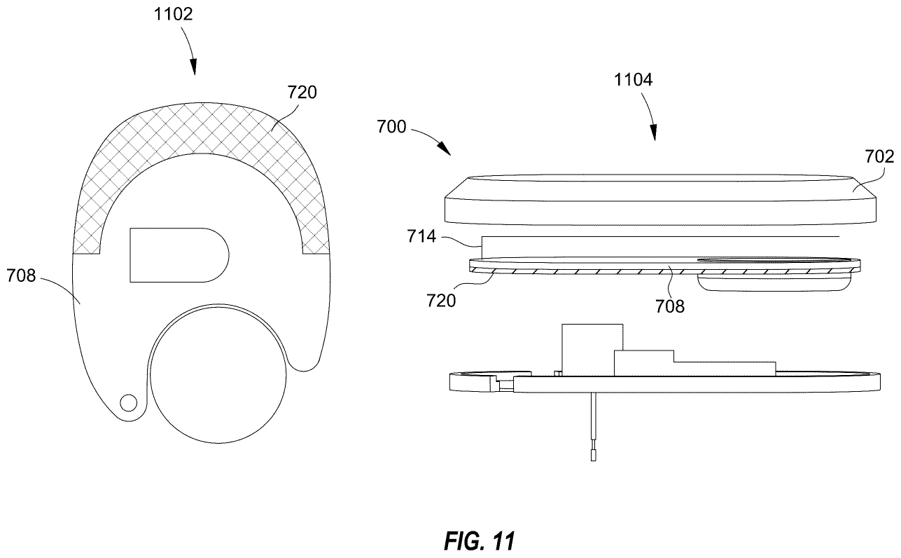

FIG. 11 illustrates another embodiment in which the mesh ground plane 720 is integrated onto the PCB 708 of the analyte sensor system 700. For example, FIG. 11 depicts a bottom view 1102 of the PCB 708 and a cross-sectional exploded view 1104 of the analyte sensor system 700. As shown in the bottom view 1102, the mesh ground plane 720 may be printed and/or formed on and/or in the PCB 708. In some cases, as shown in the cross-sectional exploded view 1104, the mesh ground plane 720 may be printed on a bottom side of the PCB 708 and the one or more antennas 714 of the analyte sensor system 700 may be located on a top side of the PCB 708. In some cases, in order to provide a sufficient amount of gain, the one or more antennas 714 may comprise a spiral antenna when the mesh ground plane 720 is printed onto the bottom side of the PCB 708.

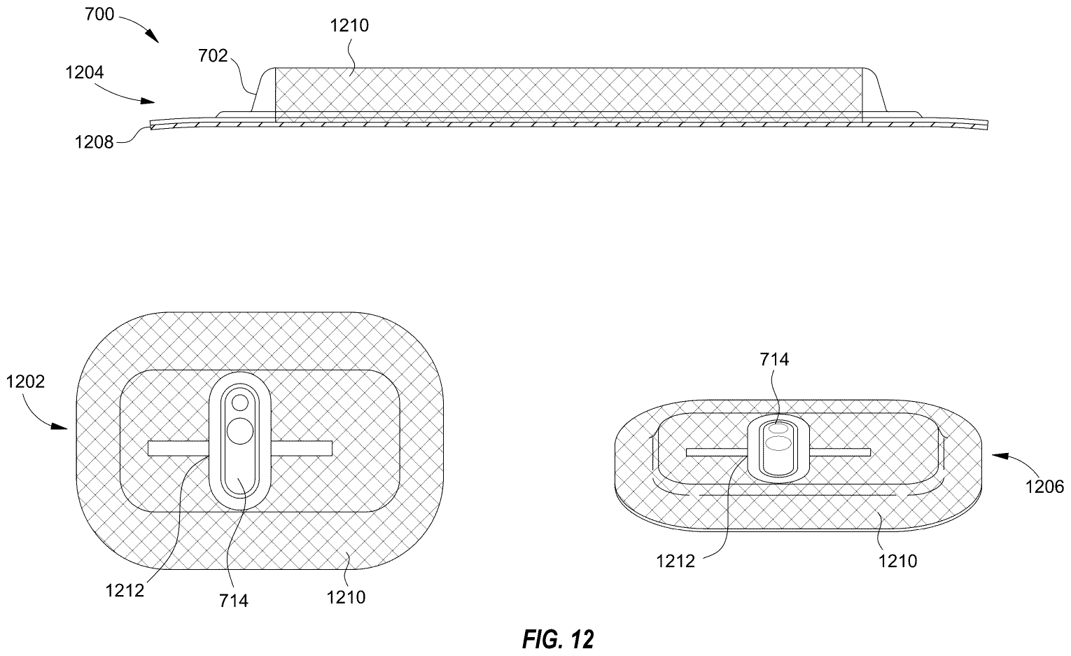

FIG. 12 illustrates another embodiment in which the analyte sensor system 700 includes a plurality of conductive mesh planes that may each, individually, be used to extend a communication/transmission range of the analyte sensor system 700. For example, FIG. 12 depicts a top view 1202 of the analyte sensor system 700, a side view 1204 of the analyte sensor system 700, and a perspective view 1206 of the analyte sensor system 700. As shown, the analyte sensor system 700 includes a first conductive mesh ground plane 1208 that is arranged similar to the mesh ground plane 720 illustrated in FIG. 7, FIG. 8, and/or FIG. 9. As with the mesh ground planes described above, the first conductive mesh ground plane 1208 may serve as an RF radiation reflector. Further, as shown, the analyte sensor system 700 includes a second conductive mesh plane 1210 that is disposed on top of the housing 702 of the analyte sensor system 700. Further, as shown, the one or more antennas 714 in the embodiment shown in FIG. 12 may include a slot antenna. In this case, the second conductive mesh plane 1210 may include a cutout 1212 for the slot antenna. Further, the second conductive mesh plane 1210 may be electrically coupled to the one or more antennas 714 (e.g., slot antenna) and may serve as a supplemental antenna or effectively as an extension of the one or more antennas 714, assisting the one or more antennas 714 in transmitting analyte data or other information. In other words, due to the electrical coupling to the one or more antennas 714, the second conductive mesh plane 1210 may act as a radiator capable of transmitting information and extending a communication/transmission range of the analyte sensor system 700.

While the techniques presented above provide a mesh ground plane improving antenna performance of next-generation analyte sensory systems, it should be understood that other types of reflective planes may be used. For example, one or more of the following may be used (1) a metallic surface that permits a first portion of radio frequencies emitted by the one or more antennas 714 to be reflected and transmitted and a second portion of the radio frequencies to be absorbed by the body of the user 704, (2) a metallic surface that reflects all of the radio frequencies emitted by the one or more antennas 714, (3) a plastic surface coated with sputtered, screened or printed metal in various patterns, or (4) a printed, stamped, chemically etched, or laser perforated metal layer in various patterns.

### Example Operations

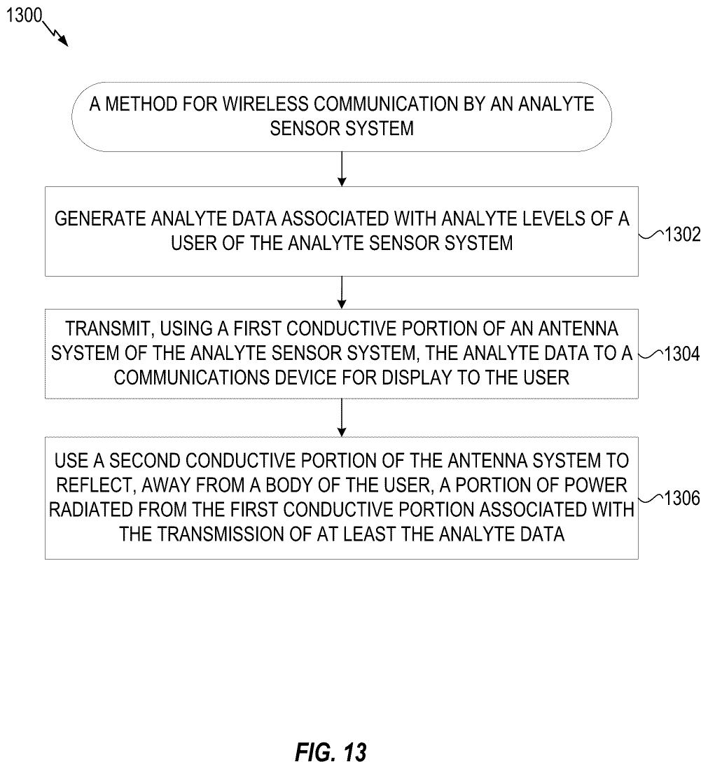

FIG. 13 shows a method 1300 for wireless communications by an analyte sensor system, such as the analyte sensor system 8 depicted and described with respect to FIG. 1, the analyte sensor system 208 depicted and described with respect to FIG. 2 and FIG. 5, and/or the analyte sensor system 700 depicted and described with respect to FIGS. 7-12.

Method 1300 begin at 1302 with the analyte sensor system generating analyte data associated with analyte levels of a user of the analyte sensor system.

At 1304, the analyte sensor system transmits, using a first conductive portion of an antenna system of the analyte sensor system, the analyte data to a communications device for display to the user; and

At 1306, the analyte sensor system uses a second conductive portion of the antenna system to reflect, away from a body of the user, a portion of power radiated from the first conductive portion associated with the transmission of at least the analyte data.

In some embodiments, the first conductive portion and a circuit board are included within a housing of the analyte sensor system.

In some embodiments, the second conductive portion is included in the housing of the analyte sensor system below the first conductive portion.

In some embodiments, the first conductive portion comprises a radiative element. In some embodiments, the second conductive portion comprises a reflective element.

In some embodiments, the second conductive portion is printed on a bottom side of the circuit board and the first conductive portion is disposed on a top side of the circuit board.

In some embodiments, the second conductive portion is disposed outside of the housing of the analyte sensor system.

In some embodiments, the second conductive portion is incorporated into an adhesive patch attached to the outside of the housing of the analyte sensor system.

In some embodiments, the second conductive portion is disposed between an adhesive patch of the analyte sensor system and the housing of the analyte sensor system.

In some embodiments, the second conductive portion is disposed on a surface of an adhesive patch configured to be attached to the body of the user.

In some embodiments, method 1300 further includes using a conductive mesh plane to assist the first conductive portion in transmitting the analyte data. In some embodiments, the conductive mesh plane is disposed on a top side of the analyte sensor system. In some embodiments, the second conductive portion is disposed on a bottom side of the analyte sensor system. In some embodiments, the conductive mesh plane is electrically coupled to the first conductive portion.

In some embodiments, the analyte sensor system includes one or more conductive contacts configured to electrically connect at least one of: the second conductive portion to the body of user; or the second conductive portion to a circuit board of the analyte sensor system.

In some embodiments, the second conductive portion includes a plurality of apertures.

In some embodiments, a size or density of the plurality of apertures is configured to tune the second conductive portion to an operating frequency defined in a wireless communication standard.

In some embodiments, the operating frequency comprises an industrial, scientific, and medical (ISM) frequency band.

In some embodiments, a size or density of the plurality of apertures is configured to allow the analyte sensor system to transmit analyte data using a bandwidth of 80 megahertz or greater.

In some embodiments, transmitting the analyte data using the first conductive portion at 1304 comprises transmitting the analyte data using Bluetooth low energy (BLE). In some embodiments, the communications device comprises a display device for displaying the analyte data to the user.

In some embodiments, the first conductive portion comprises one or more antennas. In some embodiments, the second conductive portion comprises a mesh ground plane.

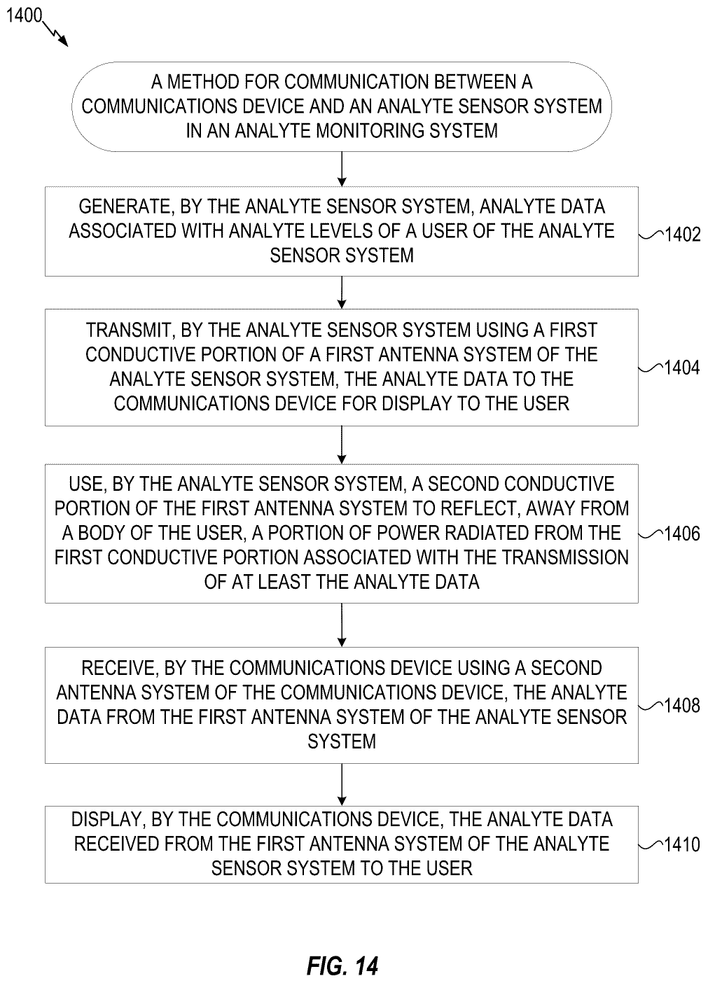

FIG. 14 shows a method for communication between a communications device and an analyte sensor system in an analyte monitoring system. In some embodiments, the analyte sensor system may be an example of the analyte sensor system 8 depicted and described with respect to FIG. 1, the analyte sensor system 208 depicted and described with respect to FIG. 2 and FIG. 5, and/or the analyte sensor system 700 depicted and described with respect to FIGS. 7-12. In some embodiments, the communications device may be an example of the display devices 110, 120, 130, and 140, partner devices 136, and/or server system 134 depicted and described with respect to FIG. 1 and/or the display device 210, the partner device 215, or the server system 234 depicted and described with respect to FIG. 2.

Method 1400 begins at 1402 the analyte sensor system generating analyte data associated with analyte levels of a user of the analyte sensor system.

At 1404, the analyte sensor system transmits, using a first conductive portion of a first antenna system of the analyte sensor system, the analyte data to the communications device for display to the user.

At 1406, the analyte sensor system uses a second conductive portion of the first antenna system to reflect, away from a body of the user, a portion of power radiated from the first conductive portion associated with the transmission of at least the analyte data.

At 1408, the communications device receives, using a second antenna system of the communications device, the analyte data from the first antenna system of the analyte sensor system.

At 1410, the communications device displays the analyte data received from the first antenna system of the analyte sensor system to the user.

In some embodiments, the first conductive portion and a circuit board are included within a housing of the analyte sensor system. In some embodiments, the second conductive portion is included in the housing of the analyte sensor system below the first conductive portion.

In some embodiments, the second conductive portion is printed on a bottom side of the circuit board and the first conductive portion is disposed on a top side of the circuit board.

In some embodiments, the first conductive portion and the circuit board are included within a housing of the analyte sensor system. In some embodiments, the second conductive portion is disposed outside of the housing of the analyte sensor system. In some embodiments, the second conductive portion is incorporated into an adhesive patch attached to the outside of the housing of the analyte sensor system. In some embodiments, the second conductive portion is disposed between an adhesive patch of the analyte sensor system and the housing of the analyte sensor system. In some embodiments, the second conductive portion is disposed on a surface of an adhesive patch configured to be attached to the body of the user.

In some embodiments, the first conductive portion and the circuit board are included within a housing of the analyte sensor system. In some embodiments, the analyte sensor system further comprises a conductive mesh plane disposed on a top side of the analyte sensor system. In some embodiments, the second conductive portion is disposed on a bottom side of the analyte sensor system. In some embodiments, the conductive mesh plane is electrically coupled to the first conductive portion and configured to assist the first conductive portion in transmitting analyte data.

In some embodiments, the second conductive portion includes a plurality of apertures. In some embodiments, a size or density of the plurality of apertures is configured to allow the analyte sensor system to transmit analyte data using a bandwidth of 80 megahertz or greater. In some embodiments, a size or density of the plurality of apertures is configured to tune the second conductive portion to an operating frequency defined in a wireless communication standard and the operating frequency comprises an industrial, scientific, and medical (ISM) frequency band.

In some embodiments, transmitting the analyte data using the first conductive portion at 1404 comprises transmitting the analyte data using Bluetooth low energy (BLE). In some embodiments, the communications device comprises a display device for displaying the analyte data to the user.

In some embodiments, the first conductive portion comprises one or more antennas. In some embodiments, the second conductive portion comprises a mesh ground plane.

### Example Health Monitoring Devices

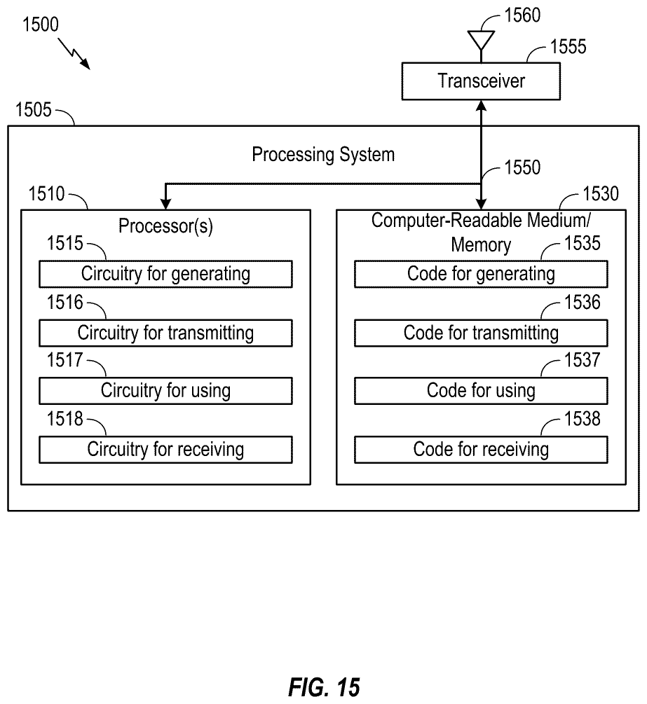

FIG. 15 depicts aspects of an example health monitoring device 1500. In some aspects, health monitoring device 1500 is an analyte sensor system, such as the analyte sensor system 8 described with respect to FIGS. 1, the analyte sensor system 208 of FIGS. 2 and 5, and/or the analyte sensor system 700 of FIGS. 7-12.

The health monitoring device 1500 includes a processing system 1505 coupled to the transceiver 1555 (e.g., a transmitter and/or a receiver). The transceiver 1555 is configured to transmit and receive signals for the health monitoring device 1500 via the first antenna system 1560, such as the various signals and messages as described herein. The processing system 1505 may be configured to perform processing functions for the health monitoring device 1500, including processing signals received and/or to be transmitted by the health monitoring device 1500.

The processing system 1505 includes one or more processors 1510. In various aspects, the one or more processors 1510 may be representative of the processor/microcontroller 535, as described with respect to FIG. 5. The one or more processors 1510 are coupled to a computer-readable medium/memory 1530 via a bus 1550. In some aspects, the computer-readable medium/memory 1530 may be representative of the storage 515, as described with respect to FIG. 2. In certain aspects, the computer-readable medium/memory 1530 is configured to store instructions (e.g., computer-executable code) that when executed by the one or more processors 1510, cause the one or more processors 1510 to perform the methods 1300 and/or 1400 described with respect to FIGS. 13 and 14, or any aspects related to these methods. Note that reference to a processor performing a function of health monitoring device 1500 may include one or more processors 1510 performing that function of health monitoring device 1500.

In the depicted example, computer-readable medium/memory 1530 stores code (e.g., executable instructions), such as code for generating 1535, code for transmitting 1536, code for using 1537, and code for receiving 1538. Processing of the code for generating 1535, code for transmitting 1536, code for using 1537, and code for receiving 1538 may cause the health monitoring device 1500 to perform the methods 1300 and/or 1400 described with respect to FIGS. 13 and 14, or any aspects related to these methods.

The one or more processors 1510 include circuitry configured to implement (e.g., execute) the code stored in the computer-readable medium/memory 1530, including circuitry for generating 1515, circuitry for transmitting 1516, circuitry for using 1517, and circuitry for receiving 1518. Processing with circuitry for generating 1515, circuitry for transmitting 1516, circuitry for using 1517, and circuitry for receiving 1518 may cause the health monitoring device 1500 to perform the methods 1300 and/or 1400 described with respect to FIGS. 13 and 14, or any aspects related to these methods.

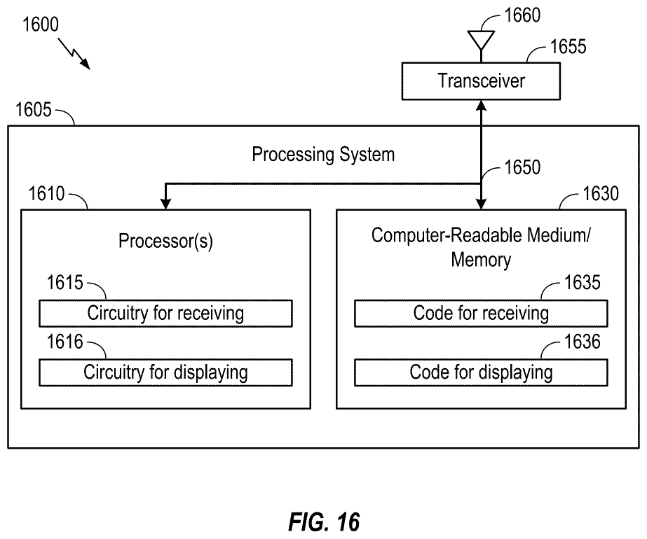

FIG. 16 depicts aspects of an example health monitoring device 1600. In some aspects, health monitoring device 1600 is a communications device, such as display devices 110, 120, 130, and 140, partner devices 136, and/or server system 134 depicted and described with respect to FIG. 1 and/or the display device 210, the partner device 215, or the server system 234 depicted and described with respect to FIG. 2.

The health monitoring device 1600 includes a processing system 1605 coupled to the transceiver 1655 (e.g., a transmitter and/or a receiver). The transceiver 1655 is configured to transmit and receive signals for the health monitoring device 1600 via the second antenna system 1660, such as the various signals and messages as described herein. The processing system 1605 may be configured to perform processing functions for the health monitoring device 1600, including processing signals received and/or to be transmitted by the health monitoring device 1600.

The processing system 1605 includes one or more processors 1610. The one or more processors 1610 are coupled to a computer-readable medium/memory 1630 via a bus 1650. In certain aspects, the computer-readable medium/memory 1630 is configured to store instructions (e.g., computer-executable code) that when executed by the one or more processors 1610, cause the one or more processors 1610 to perform the method 1400 described with respect to FIG. 14, or any aspect related to this method. Note that reference to a processor performing a function of health monitoring device 1600 may include one or more processors 1610 performing that function of health monitoring device 1600.

In the depicted example, computer-readable medium/memory 1630 stores code (e.g., executable instructions), such as code for receiving 1635 and code for displaying 1636. Processing of the code for receiving 1635 and code for displaying 1636 may cause the health monitoring device 1600 to perform the method 1400 described with respect to FIG. 14, or any aspect related to this method.

The one or more processors 1610 include circuitry configured to implement (e.g., execute) the code stored in the computer-readable medium/memory 1630, including circuitry for receiving 1615 and circuitry for displaying 1616. Processing with circuitry for receiving 1615 and circuitry for displaying 1616 may cause the health monitoring device 1600 to perform the method 1400 described with respect to FIG. 14, or any aspect related to this method.

### Example Clauses

Implementation examples are described in the following numbered clauses:

Clause 1: An analyte sensor system, comprising: an analyte sensor configured to generate analyte data associated with analyte levels of a user of the analyte sensor system; a first conductive portion configured to transmit the analyte data to a communications device; a circuit board configured to operatively connect the analyte sensor with the first conductive portion; and a second conductive portion configured to reflect, away from a body of the user, a portion of power radiated from the first conductive portion associated with transmission of at least the analyte data.

Clause 2: The analyte sensor system of Clause 1, wherein the first conductive portion and the circuit board are included within a housing of the analyte sensor system.

Clause 3: The analyte sensor system of Clause 2, wherein the second conductive portion is included in the housing of the analyte sensor system below the first conductive portion.

Clause 4: The analyte sensor system of Clause 3, wherein: the first conductive portion comprises a radiative element; and the second conductive portion comprises a reflective element.

Clause 5: The analyte sensor system of Clause 3, wherein the second conductive portion is printed on a bottom side of the circuit board and the first conductive portion is disposed on a top side of the circuit board.

Clause 6: The analyte sensor system of Clause 2, wherein the second conductive portion is disposed outside of the housing of the analyte sensor system.

Clause 7: The analyte sensor system of Clause 6, wherein the second conductive portion is incorporated into an adhesive patch attached to the outside of the housing of the analyte sensor system.

Clause 8: The analyte sensor system of Clause 6, wherein the second conductive portion is disposed between an adhesive patch of the analyte sensor system and the housing of the analyte sensor system.

Clause 9: The analyte sensor system of Clause 6, wherein the second conductive portion is disposed on a surface of an adhesive patch configured to be attached to the body of the user.

Clause 10: The analyte sensor system of any one of Clauses 2-9, further comprising a conductive mesh plane disposed on a top side of the analyte sensor system, wherein: the second conductive portion is disposed on a bottom side of the analyte sensor system; and the conductive mesh plane is electrically coupled to the first conductive portion and configured to assist the first conductive portion in transmitting analyte data.

Clause 11: The analyte sensor system of any one of Clauses 1-10, further comprising one or more conductive contacts configured to electrically connect at least one of: the second conductive portion to the body of user; or the second conductive portion to the circuit board.

Clause 12: The analyte sensor system of any one of Clauses 1-11, wherein the second conductive portion includes a plurality of apertures.

Clause 13: The analyte sensor system of Clause 12, wherein a size or density of the plurality of apertures is configured to tune the second conductive portion to an operating frequency defined in a wireless communication standard.

Clause 14: The analyte sensor system of Clause 13, wherein the operating frequency comprises an industrial, scientific, and medical (ISM) frequency band.

Clause 15: The analyte sensor system of any one of Clauses 12-14, wherein a size or density of the plurality of apertures is configured to allow the analyte sensor system to transmit analyte data using a bandwidth of 80 megahertz or greater.

Clause 16: The analyte sensor system of any one of Clauses 1-15, wherein: the first conductive portion is configured to transmit the analyte data to the communications device using Bluetooth low energy (BLE); and the communications device comprises a display device for displaying the analyte data to the user.

Clause 17: The analyte sensor system of any one of Clauses 1-16, wherein: the first conductive portion comprises one or more antennas; and the second conductive portion comprises a mesh ground plane.

Clause 18: An antenna system for communicating analyte data, comprising: a first conductive portion operatively coupled to an analyte sensor via a circuit board, wherein the first conductive portion is configured to: receive analyte data associated with analyte levels of a user of an analyte sensor system; and transmit the analyte data to a communications device for display to the user; and a second conductive portion coupled with the circuit board, wherein the second conductive portion is configured to reflect, away from a body of the user, a portion of power radiated from the first conductive portion associated with transmission of at least the analyte data.

Clause 19: The antenna system of Clause 18, wherein the first conductive portion and the circuit board are included within a housing of the analyte sensor system.

Clause 20: The antenna system of Clause 19, wherein the second conductive portion is included in the housing of the analyte sensor system below the first conductive portion.

Clause 21: The antenna system of Clause 20, wherein: the first conductive portion comprises a radiative element; and the second conductive portion comprises a reflective element.

Clause 22: The antenna system of Clause 20, wherein the second conductive portion is printed on a bottom side of the circuit board and the first conductive portion is disposed on a top side of the circuit board.

Clause 23: The antenna system of Clause 19, wherein the second conductive portion is disposed outside of the housing of the analyte sensor system.

Clause 24: The antenna system of Clause 23, wherein the second conductive portion is incorporated into an adhesive patch attached to the outside of the housing of the analyte sensor system.

Clause 25: The antenna system of Clause 23, wherein the second conductive portion is disposed between an adhesive patch of the analyte sensor system and the housing of the analyte sensor system.

Clause 26: The antenna system of Clause 23, wherein the second conductive portion is disposed on a surface of an adhesive patch configured to be attached to the body of the user.

Clause 27: The antenna system of any one of Clauses 19-26, further comprising a conductive mesh plane disposed on a top side of the analyte sensor system, wherein: the second conductive portion is disposed on a bottom side of the analyte sensor system; and the conductive mesh plane is electrically coupled to the first conductive portion and configured to assist the first conductive portion in transmitting analyte data.

Clause 28: The antenna system of any one of Clauses 18-27, wherein the second conductive portion includes a plurality of apertures.

Clause 29: The antenna system of Clause 28, wherein: a size or density of the plurality of apertures is configured to tune the second conductive portion to an operating frequency defined in a wireless communication standard; and the operating frequency comprises an industrial, scientific, and medical (ISM) frequency band.

Clause 30: The antenna system of any one of Clauses 28-29, wherein a size or density of the plurality of apertures is configured to allow the analyte sensor system to transmit analyte data using a bandwidth of 80 megahertz or greater.

Clause 31: The antenna system of any one of Clauses 18-30, wherein: the first conductive portion is configured to transmit the analyte data to the communications device using Bluetooth low energy (BLE); and the communications device comprises a display device for displaying the analyte data to the user.

Clause 32: The analyte sensor system of any one of Clauses 18-31, wherein: the first conductive portion comprises one or more antennas; and the second conductive portion comprises a mesh ground plane.

Clause 33: An analyte monitoring system, comprising: a communications device; and an analyte sensor system comprising: an analyte sensor configured to generate analyte data associated with analyte levels of a user of the analyte sensor system; a first antenna system comprising: a first conductive portion configured to receive the analyte data from the analyte sensor and to transmit the analyte data to the communications device for display to the user; and a second conductive portion configured to reflect, away from a body of the user, a portion of power radiated from the first conductive portion associated with transmission of at least the analyte data; and a circuit board configured to operatively connect the analyte sensor with the first conductive portion, wherein: the communications device comprises a second antenna system configured to receive the analyte data from the first antenna system of the analyte sensor system; and the communications device is configured to display the analyte data received from the first antenna system of the analyte sensor system to the user.

Clause 34: The analyte monitoring system of Clause 33, wherein: the first conductive portion and the circuit board are included within a housing of the analyte sensor system; and the second conductive portion is included in the housing of the analyte sensor system below the first conductive portion.

Clause 35: The analyte monitoring system of Clause 34, wherein the second conductive portion is printed on a bottom side of the circuit board and the first conductive portion is disposed on a top side of the circuit board.

Clause 36: The analyte monitoring system of Clause 34, wherein: the first conductive portion and the circuit board are included within a housing of the analyte sensor system; the second conductive portion is disposed outside of the housing of the analyte sensor system; and one of: the second conductive portion is incorporated into an adhesive patch attached to the outside of the housing of the analyte sensor system; the second conductive portion is disposed between an adhesive patch of the analyte sensor system and the housing of the analyte sensor system; or the second conductive portion is disposed on a surface of an adhesive patch configured to be attached to the body of the user.

Clause 37: The analyte monitoring system of Clause 34, wherein: the first conductive portion and the circuit board are included within a housing of the analyte sensor system; the analyte sensor system further comprises a conductive mesh plane disposed on a top side of the analyte sensor system; the second conductive portion is disposed on a bottom side of the analyte sensor system; and the conductive mesh plane is electrically coupled to the first conductive portion and configured to assist the first conductive portion in transmitting analyte data.

Clause 38: The analyte monitoring system of any one of Clauses 33-37, wherein: the second conductive portion includes a plurality of apertures; and at least one of: a size or density of the plurality of apertures is configured to allow the analyte sensor system to transmit analyte data using a bandwidth of 80 megahertz or greater; or a size or density of the plurality of apertures is configured to tune the second conductive portion to an operating frequency defined in a wireless communication standard and the operating frequency comprises an industrial, scientific, and medical (ISM) frequency band.

Clause 39: The analyte monitoring system of any one of Clauses 33-38, wherein: the first conductive portion is configured to transmit the analyte data to the communications device using Bluetooth low energy (BLE); and the communications device comprises a display device for displaying the analyte data to the user.

Clause 40: The analyte monitoring system of any one of Clauses 33-39, wherein: the first conductive portion comprises one or more antennas; and the second conductive portion comprises a mesh ground plane.

Clause 41: A method for wireless communication by an analyte sensor system, comprising: generating analyte data associated with analyte levels of a user of the analyte sensor system; transmitting, using a first conductive portion of an antenna system of the analyte sensor system, the analyte data to a communications device for display to the user; and using a second conductive portion of the antenna system to reflect, away from a body of the user, a portion of power radiated from the first conductive portion associated with the transmission of at least the analyte data.

Clause 42: The method of Clause 41, wherein the first conductive portion and a circuit board are included within a housing of the analyte sensor system.

Clause 43: The method of Clause 42, wherein the second conductive portion is included in the housing of the analyte sensor system below the first conductive portion.

Clause 44: The method of Clause 43, wherein: the first conductive portion comprises a radiative element; and the second conductive portion comprises a reflective element.

Clause 45: The method of any one of Clauses 43-44, wherein the second conductive portion is printed on a bottom side of the circuit board and the first conductive portion is disposed on a top side of the circuit board.

Clause 46: The method of Clause 42, wherein the second conductive portion is disposed outside of the housing of the analyte sensor system.

Clause 47: The method of Clause 46, wherein the second conductive portion is incorporated into an adhesive patch attached to the outside of the housing of the analyte sensor system.

Clause 48: The method of Clause 46, wherein the second conductive portion is disposed between an adhesive patch of the analyte sensor system and the housing of the analyte sensor system.

Clause 49: The method of Clause 46, wherein the second conductive portion is disposed on a surface of an adhesive patch configured to be attached to the body of the user.

Clause 50: The method of Clause 42, further comprising using a conductive mesh plane to assist the first conductive portion in transmitting the analyte data, wherein: the conductive mesh plane is disposed on a top side of the analyte sensor system; the second conductive portion is disposed on a bottom side of the analyte sensor system; and the conductive mesh plane is electrically coupled to the first conductive portion.

Clause 51: The method of any one of Clauses 41-50, wherein the analyte sensor system includes one or more conductive contacts configured to electrically connect at least one of: the second conductive portion to the body of user; or the second conductive portion to a circuit board of the analyte sensor system.

Clause 52: The method of any one of Clauses 41-52, wherein the second conductive portion includes a plurality of apertures.

Clause 53: The method of Clause 52, wherein a size or density of the plurality of apertures is configured to tune the second conductive portion to an operating frequency defined in a wireless communication standard.

Clause 54: The method of Clause 53, wherein the operating frequency comprises an industrial, scientific, and medical (ISM) frequency band.

Clause 55: The method of Clause 52, wherein a size or density of the plurality of apertures is configured to allow the analyte sensor system to transmit analyte data using a bandwidth of 80 megahertz or greater.

Clause 56: The method of any one of Clauses 41-55, wherein: transmitting the analyte data using the first conductive portion comprises transmitting the analyte data using Bluetooth low energy (BLE); and the communications device comprises a display device for displaying the analyte data to the user.

Clause 57: The method of any one of Clauses 41-56, wherein: the first conductive portion comprises one or more antennas; and the second conductive portion comprises a mesh ground plane.

Clause 58: A method for communication between a communications device and an analyte sensor system in an analyte monitoring system, comprising: generating, by the analyte sensor system, analyte data associated with analyte levels of a user of the analyte sensor system; transmitting, by the analyte sensor system using a first conductive portion of a first antenna system of the analyte sensor system, the analyte data to the communications device for display to the user; using, by the analyte sensor system, a second conductive portion of the first antenna system to reflect, away from a body of the user, a portion of power radiated from the first conductive portion associated with the transmission of at least the analyte data; receiving, by the communications device using a second antenna system of the communications device, the analyte data from the first antenna system of the analyte sensor system; and displaying, by the communications device, the analyte data received from the first antenna system of the analyte sensor system to the user.

Clause 59: The method of Clause 58, wherein: the first conductive portion and a circuit board are included within a housing of the analyte sensor system; and the second conductive portion is included in the housing of the analyte sensor system below the first conductive portion.

Clause 60: The method of Clause 59, wherein the second conductive portion is printed on a bottom side of the circuit board and the first conductive portion is disposed on a top side of the circuit board.

Clause 61: The method of Clause 59, wherein: the first conductive portion and the circuit board are included within a housing of the analyte sensor system; the second conductive portion is disposed outside of the housing of the analyte sensor system; and one of: the second conductive portion is incorporated into an adhesive patch attached to the outside of the housing of the analyte sensor system; the second conductive portion is disposed between an adhesive patch of the analyte sensor system and the housing of the analyte sensor system; or the second conductive portion is disposed on a surface of an adhesive patch configured to be attached to the body of the user.

Clause 62: The method of Clause 59, wherein: the first conductive portion and the circuit board are included within a housing of the analyte sensor system; the analyte sensor system further comprises a conductive mesh plane disposed on a top side of the analyte sensor system; the second conductive portion is disposed on a bottom side of the analyte sensor system; and the conductive mesh plane is electrically coupled to the first conductive portion and configured to assist the first conductive portion in transmitting analyte data.

Clause 63: The method of any one of Clauses 58-62, wherein: the second conductive portion includes a plurality of apertures; and at least one of: a size or density of the plurality of apertures is configured to allow the analyte sensor system to transmit analyte data using a bandwidth of 80 megahertz or greater; or a size or density of the plurality of apertures is configured to tune the second conductive portion to an operating frequency defined in a wireless communication standard and the operating frequency comprises an industrial, scientific, and medical (ISM) frequency band.

Clause 64: The method of any one of Clauses 58-63, wherein: transmitting the analyte data using the first conductive portion comprises transmitting the analyte data using Bluetooth low energy (BLE); and the communications device comprises a display device for displaying the analyte data to the user.

Clause 65: The method of any one of Clauses 58-64, wherein: the first conductive portion comprises one or more antennas; and the second conductive portion comprises a mesh ground plane.

Clause 66: An apparatus, comprising: a memory comprising executable instructions; and one or more processors configured to execute the executable instructions and cause the apparatus to perform a method in accordance with any one of Clauses 41-65.

Clause 67: An apparatus, comprising means for performing a method in accordance with any one of Clauses 41-65.

Clause 68: A non-transitory computer-readable medium comprising executable instructions that, when executed by one or more processors of an apparatus, cause the apparatus to perform a method in accordance with any one of Clauses 41-65.

Clause 69: A computer program product embodied on a computer-readable storage medium comprising code for performing a method in accordance with any one of Clauses 41-65.

### Additional Considerations

In this document, the terms “computer program medium” and “computer usable medium” and “computer readable medium”, as well as variations thereof, are used to generally refer to transitory or non-transitory media. These and other various forms of computer program media or computer usable/readable media may be involved in carrying one or more sequences of one or more instructions to a processing device for execution. Such instructions embodied on the medium, may generally be referred to as “computer program code” or a “computer program product” or “instructions” (which may be grouped in the form of computer programs or other groupings). When executed, such instructions may enable a computing module, such as the analyte sensor system 8, the analyte sensor 208, the display device 210, circuitry related thereto, and/or a processor thereof or connected thereto to perform features or functions of the present disclosure as discussed herein (for example, in connection with methods described above and/or in the claims), including for example when the same is/are incorporated into a system, apparatus, device and/or the like.

Various embodiments have been described with reference to specific example features thereof. It will, however, be evident that various modifications and changes may be made thereto without departing from the broader spirit and scope of the various embodiments as set forth in the appended claims. The specification and figures are, accordingly, to be regarded in an illustrative rather than a restrictive sense. It will be appreciated that, for clarity purposes, the above description has described embodiments with reference to different functional units. However, it will be apparent that any suitable distribution of functionality between different functional units may be used without detracting from the invention. For example, functionality illustrated to be performed by separate computing devices may be performed by the same computing device. Likewise, functionality illustrated to be performed by a single computing device may be distributed amongst several computing devices. Hence, references to specific functional units are only to be seen as references to suitable means for providing the described functionality, rather than indicative of a strict logical or physical structure or organization.

Although described above in terms of various example embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the present application, whether or not such embodiments are described and whether or not such features are presented as being a part of a described embodiment. Thus, the breadth and scope of the present application should not be limited by any of the above-described example embodiments.

Terms and phrases used in the present application, and variations thereof, unless otherwise expressly stated, should be construed as open ended as opposed to limiting. As examples of the foregoing: the term “including” should be read as meaning “including, without limitation” or the like; the term “example” is used to provide illustrative instances of the item in discussion, not an exhaustive or limiting list thereof; the terms “a” or “an” should be read as meaning “at least one,” “one or more” or the like; the term “set” should be read to include one or more objects of the type included in the set; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Similarly, the plural may in some cases be recognized as applicable to the singular and vice versa. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic, circuitry, or other components, may be combined in a single package or separately maintained and may further be distributed in multiple groupings or packages or across multiple locations.

Additionally, the various embodiments set forth herein are described in terms of example block diagrams, flow charts, and other illustrations. As will become apparent to one of ordinary skill in the art after reading this document, the illustrated embodiments and their various alternatives may be implemented without confinement to the illustrated examples. For example, block diagrams and their accompanying description should not be construed as mandating a particular architecture or configuration. Moreover, the operations and sub-operations of various methods described herein are not necessarily limited to the order described or shown in the figures, and one of skill in the art will appreciate, upon studying the present disclosure, variations of the order of the operations described herein that are within the spirit and scope of the disclosure. It will be understood that each block of the flowchart illustrations, and combinations of blocks in the flowchart illustrations, can be implemented by execution of computer program instructions. These computer program instructions may be loaded onto a computer or other programmable data processing apparatus (such as a controller, microcontroller, microprocessor or the like) in a sensor electronics system to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks presented herein.

It should be appreciated that all methods and processes disclosed herein may be used in any glucose or other analyte monitoring system, continuous or intermittent, or any device with or without an analyte sensor that is capable of providing vital health data associated with a user. It should further be appreciated that the implementation and/or execution of all methods and processes may be performed by any suitable devices or systems, whether local or remote. Further, any combination of devices or systems may be used to implement the present methods and processes.

In addition, the operations and sub-operations of methods described herein may be carried out or implemented, in some cases, by one or more of the components, elements, devices, modules, circuitry, processors, etc. of systems, apparatuses, devices, environments, and/or computing modules described herein and referenced in various of figures of the present disclosure, as well as one or more sub-components, elements, devices, modules, processors, circuitry, and the like depicted therein and/or described with respect thereto. In such instances, the description of the methods or aspects thereof may refer to a corresponding component, element, etc., but regardless of whether an explicit reference is made, one of skill in the art will recognize upon studying the present disclosure when the corresponding component, element, etc. may be used. Further, it will be appreciated that such references do not necessarily limit the described methods to the particular component, element, etc. referred to. Thus, it will be appreciated by one of skill in the art that aspects and features described above in connection with (sub-) components, elements, devices, modules, and circuitry, etc., including variations thereof, may be applied to the various operations described in connection with methods described herein, and vice versa, without departing from the scope of the present disclosure.

1. An analyte sensor system, comprising:
an analyte sensor configured to generate analyte data associated with analyte levels of a user of the analyte sensor system;
a first conductive portion configured to transmit the analyte data to a communications device;
a circuit board configured to operatively connect the analyte sensor with the first conductive portion; and
a second conductive portion configured to reflect, away from a body of the user, a portion of power radiated from the first conductive portion associated with transmission of at least the analyte data.

2. The analyte sensor system of claim 1, wherein the first conductive portion and the circuit board are included within a housing of the analyte sensor system.

3. The analyte sensor system of claim 2, wherein the second conductive portion is included in the housing of the analyte sensor system below the first conductive portion.

4. The analyte sensor system of claim 3, wherein:
the first conductive portion comprises a radiative element; and
the second conductive portion comprises a reflective element.

5. The analyte sensor system of claim 3, wherein the second conductive portion is printed on a bottom side of the circuit board and the first conductive portion is disposed on a top side of the circuit board.

6. The analyte sensor system of claim 2, wherein the second conductive portion is disposed outside of the housing of the analyte sensor system.

7. The analyte sensor system of claim 6, wherein the second conductive portion is incorporated into an adhesive patch attached to the outside of the housing of the analyte sensor system.

8. The analyte sensor system of claim 6, wherein the second conductive portion is disposed between an adhesive patch of the analyte sensor system and the housing of the analyte sensor system.

9. The analyte sensor system of claim 6, wherein the second conductive portion is disposed on a surface of an adhesive patch configured to be attached to the body of the user.

10. The analyte sensor system of claim 2, further comprising a conductive mesh plane disposed on a top side of the analyte sensor system, wherein:
the second conductive portion is disposed on a bottom side of the analyte sensor system; and
the conductive mesh plane is electrically coupled to the first conductive portion and configured to assist the first conductive portion in transmitting analyte data.

11. The analyte sensor system of claim 1, further comprising one or more conductive contacts configured to electrically connect at least one of:
the second conductive portion to the body of user; or
the second conductive portion to the circuit board.

12. The analyte sensor system of claim 1, wherein the second conductive portion includes a plurality of apertures.

13. The analyte sensor system of claim 12, wherein a size or density of the plurality of apertures is configured to tune the second conductive portion to an operating frequency defined in a wireless communication standard.

14. The analyte sensor system of claim 13, wherein the operating frequency comprises an industrial, scientific, and medical (ISM) frequency band.

15. The analyte sensor system of claim 12, wherein a size or density of the plurality of apertures is configured to allow the analyte sensor system to transmit analyte data using a bandwidth of 80 megahertz or greater.

16. The analyte sensor system of claim 1, wherein:
the first conductive portion is configured to transmit the analyte data to the communications device using Bluetooth low energy (BLE); and
the communications device comprises a display device for displaying the analyte data to the user.

17. The analyte sensor system of claim 1, wherein:
the first conductive portion comprises one or more antennas; and
the second conductive portion comprises a mesh ground plane.

18. An antenna system for communicating analyte data, comprising:
a first conductive portion operatively coupled to an analyte sensor via a circuit board, wherein the first conductive portion is configured to:
receive analyte data associated with analyte levels of a user of an analyte sensor system; and
transmit the analyte data to a communications device for display to the user; and
a second conductive portion coupled with the circuit board, wherein the second conductive portion is configured to reflect, away from a body of the user, a portion of power radiated from the first conductive portion associated with transmission of at least the analyte data.

19-31. (canceled)

32. The analyte sensor system of claim 18, wherein:
the first conductive portion comprises one or more antennas; and
the second conductive portion comprises a mesh ground plane.

33. An analyte monitoring system, comprising:
a communications device; and
an analyte sensor system comprising:
an analyte sensor configured to generate analyte data associated with analyte levels of a user of the analyte sensor system;
a first antenna system comprising:
a first conductive portion configured to receive the analyte data from the analyte sensor and to transmit the analyte data to the communications device for display to the user; and
a second conductive portion configured to reflect, away from a body of the user, a portion of power radiated from the first conductive portion associated with transmission of at least the analyte data; and
a circuit board configured to operatively connect the analyte sensor with the first conductive portion, wherein:
the communications device comprises a second antenna system configured to receive the analyte data from the first antenna system of the analyte sensor system; and
the communications device is configured to display the analyte data received from the first antenna system of the analyte sensor system to the user.

34-65. (canceled)
